(19) United States
(12) Patent Application Publication
(10) Pub. No.: US 2021/0088512 A1
(43) Pub. Date: Mar. 25, 2021

(54) POINT OF-CARE DIAGNOSTICS BASED ON A CHANGE IN PARTICLE MOTION BEHAVIOR

(71) Applicant: THE BRIGHAM AND WOMEN'S HOSPITAL, INC., Boston, MA (US)

(72) Inventors: Mohamed Shehata Draz, Boston, MA (US); Hadi Hadi, Boston, MA (US)

(21) Appl. No.: 17/052,569

(22) PCT Filed: May 3, 2019
(86) PCT No.: PCT/US19/30540

§ 371 Date: Nov. 3, 2020

Related U.S. Application Data
(60) Provisional application No. 62/666,309, filed on May 3, 2018.

(51) Int. Cl. G01N 33/543; B01L 3/00; G01N 33/569; G01N 15/00

(52) U.S. Cl. CPC G01N 33/54306; B01L 3/502715; G01N 33/54346; G01N 33/56983; H04M 1/724; B01L 2200/10; B01L 2300/06; G01N 2333/185; G01N 2015/0003; G01N 15/00

(57) ABSTRACT

A system that monitors particle motion behavior for point-of-care diagnostics is described. The system can include a sample testing unit configured to house a sample. The sample testing unit can include a plurality of motor structures configured for self-propulsion based on a presence or an absence of a target analyte in the sample and a plurality of beads configured to experience a motion behavior based on the self-propulsion of the plurality of motor structures. Each of the plurality of motor structures can include a catalytic motor-like micro/nanoparticle; and an attached functional material specific for the target analyte attached to the catalytic motor-like particle. The optical recording unit can include an optical arrangement configured to detect the motion behavior of the beads in the sample testing unit. The motion behavior can be indicative of the presence or the absence of the target analyte.

### RELATED APPLICATIONS

This application claims the benefit of U.S. Provisional Application No. 62/666,309, filed May 3, 2018, entitled “NUCLEIC ACID PAYLOAD AND PARTICLE MOTION FOR POINT OF CARE DIAGNOSTICS,” the entirety of which is hereby incorporated by reference for all purposes.

### TECHNICAL FIELD

The present disclosure relates generally point-of-care diagnostics and more specifically to systems and methods that monitor particle motion behavior for point-of-care diagnostics.

### BACKGROUND

Motion-based biosensors that employ self-propelling catalytic motor structures have attracted considerable attention in medicine and engineering because of their potential for real-time use at a high spatial resolution. Generally, the motor structures can include micro/nanoparticles each with an attached functional material. The micro/nanoparticles possess catalytic properties and can employ these catalytic properties to become self-propelling catalytic motor-like structures that convert chemical energy into mechanical motion (e.g., via self-electrophoresis, self-diffusiophoresis, bubble thrust, or the like) that is autonomous, powerful, remotely controlled, and/or ultrafast. The functional materials can be attached to the micro/nanoparticles during fabrication of the micro/nanoparticles and/or as a surface modification of the micro/nanoparticles. Such self-propelling catalytic motor structures have been used in chemical and biological sensing, drug delivery, controlled transport and release of biomolecules, cell screening and manipulation, and waste treatment. Biological sensing applications, in particular, have related to the use of motion-based biosensors for the detection of nucleic acid and protein targets. Although these biosensors have demonstrated good performance and potential for target detection, the biosensors require sophisticated optical microscopy systems to track the motion and speed of motor structures in the presence of target analyte, making these biosensors impractical for point-of-care diagnostics.

### SUMMARY

Described herein is a solution that makes biosensors that rely on the motion and speed of motor structures practical for point-of-care diagnostics.

An aspect of the present disclosure relates to a system that monitors particle motion behavior for point-of-care diagnostics. The system can include a sample testing unit that houses a sample and an optical recording unit. The sample testing unit can include a plurality of motor structures configured for self-propulsion based on a presence or an absence of a target analyte in the sample and a plurality of beads configured to experience a motion behavior based on the self-propulsion of the plurality of motor structures. Each of the plurality of motor structures includes a catalytic motor-like micro/nanoparticle and an attached functional material specific for the target analyte. The optical recording unit includes an optical arrangement configured to detect the motion behavior of the beads in the sample testing unit. The motion behavior is indicative of the presence or the absence of the target analyte.

Another aspect of the present disclosure relates to a method for monitoring particle motion behavior for point-of-care diagnostics. A sample can be loaded into an optical attachment of a handheld device, which includes a processor. The sample includes a plurality of motor structures configured for self-propulsion based on a presence or an absence of a target analyte in the sample and a plurality of beads. The handheld device can determine an initial motion characteristic of the plurality of beads within the sample and track a change from the initial motion characteristic of the plurality of beads within the sample. The change from the initial motion characteristic can be based on the presence or the absence of the target analyte in the sample.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram showing an example of a system that monitors particle motion behavior for point-of-care diagnostics in accordance with an aspect of the present disclosure;

FIG. 2 is a diagram showing an example of bead and a motor structure that can be used within the system of FIG. 1;

FIG. 3 is a diagram showing a zoomed in example of the motor structure of FIG. 2;

FIG. 4 is a process flow diagrams showing an example method for monitoring particle motion behavior for point-of-care diagnostics according to an aspect of the present disclosure;

FIG. 5 shows an example operation of a nanomotor-based bead-motion cellphone (NBC) system loaded with a sample;

FIG. 6 shows different stages of an example of a motion tracking application interface and data processing;

FIG. 7 shows a schematic presentation of a protocol used in the preparation of Pt-nanomotors (also referred to as Pt-nanoprobes);

FIG. 8 shows a transmission electron microscopy (TEM) micrograph and particle size distribution histogram of the prepared Pt-nanoparticles (PtNPs);

FIG. 9 shows FT-IR spectra of the prepared Pt-nanomotors;

FIG. 10 shows a UV-Vis absorption spectra of PtNPs and the prepared Pt-nanomotors;

FIG. 11 shows agarose gel electrophoresis of PtNPs and Pt-nanomotors;

FIG. 12 shows a schematic presentation of surface medication of polystyrene (PS) beads with anti-Zika virus (ZIKV) monoclonal antibody (mAb);

FIG. 13 shows UV-vis absorption values at 223 nm for beads with and without antibody modification;

FIG. 14 shows FT-IR spectra of the prepared anti-ZIKV mAb modified beads;

FIG. 15 shows SDS gel electrophoresis for ZIKV captured on the surface of antibody-modified beads;

FIG. 16 shows SEM analysis of beads with Pt-virus complexes;

FIG. 17 shows motion analysis of beads in the presence and absence of the target ZIKV tested under bright-field light microscopy using 200× magnification power;

FIG. 18 shows representative images of motion trajectories of beads in the presence and absence of ZIKV under light microscopy;

FIG. 19 shows the change in bead motion magnitude in the presence of different concentrations of ZIKV;

FIG. 20 shows trajectory images of the motion of beads in the control sample and samples with different concentrations of ZIKV;

FIG. 21 shows the change in bead motion magnitude due to ZIKV and non-target viruses;

FIG. 22 shows trajectory images of the motion of beads in the presence of ZIKV and other non-target viruses;

FIG. 23 shows detection of ZIKV spiked in urine samples with different virus concentrations;

FIG. 24 shows trajectory images of beads motion recorded for ZIKV-spiked urine samples;

FIG. 25 shows detection of ZIKV-spiked saliva samples;

FIG. 26 shows trajectory images of beads motion recorded for ZIKV-spiked saliva samples;

FIG. 27 shows a schematic diagram of HIV-1 detection using a system that integrates cellphone-based optical sensing, loop mediated isothermal amplification and micrometer motion (CALM);

FIG. 28 shows a schematic of a motor preparation reaction;

FIG. 29 shows TEM images for prepared AuNPs (left, scale bar=200 nm) and PtNPs (right, scale bar=10 nm);

FIG. 30 shows digital images of PtNPs and AuNPs used in micromotor preparation and corresponding UV-vis absorbance spectra of the as-prepared nanoparticle solutions;

FIG. 31 shows FT-IR analysis of AuNPs with and without a DNA capture probe;

FIG. 32 shows UV-vis absorbance spectra of the as-prepared nanoparticle solutions;

FIG. 33 shows silver staining reaction results confirming stable addition of AuNP-modified DNA to the surface of PS beads for motor preparation;

FIG. 34 shows DNA-AuNP beads after addition of PtNPs;

FIG. 35 shows agarose gel electrophoresis confirming the presence of DNA capture probe on the surface of the prepared motors using a synthetic target DNA;

FIG. 36 shows fluorescence imaging of LAMP amplicons captured and isolated using the prepared motors confirming the full activity of the motors to specifically interact with the target DNA amplicons;

FIG. 37 shows motion of 6 μm motors in solutions with different concentrations of hydrogen peroxide (H₂O₂) (scale bar=50 μm);

FIG. 38 shows thermal stability of motors at different temperatures tested in 5% H₂O₂ solution;

FIG. 39 shows stability of motor motion with time and the digital image shows the motion trajectories of 6 mm motors in 15% H₂O₂ solution after 30 s (scale bar=50 μm);

FIG. 40 shows motion trajectories of 6 μm motors with and without H₂O₂;

FIG. 41 shows mean squared displacement (MSD) plotted against time (t) for motor motion in 0% H₂O₂ (left) or 5% H₂O₂ (right);

FIG. 42 shows motion trajectories of a mixture of 3 μm beads (no PtNPs or AuNPs were added) and 6 μm motors (beads with PtNPs and AuNPs);

FIG. 43 shows MSD analysis of a mixture of shows motion trajectories of a mixture of 3 μm beads (no PtNPs or AuNPs, left) and 6 μm motors (beads with PtNPs and AuNPs, right);

FIG. 44 shows operating the CALM system with a cellphone optical accessory and a disposable microchip;

FIG. 45 shows an exploded 3D schematic of the cellphone attachment, including a casting stage with optics, a sample holder, and a back cover with LED

FIG. 46 shows a motion tracking application that is used to detect and measure the motion of DNA-motors in H₂O₂ solution;

FIG. 47 shows a correlation between the performance of the motion tracking application and bright-field light microscopy coupled with the ImageJ software in velocity detection of different motor samples (n=50);

FIG. 48 shows agarose gel electrophoresis image of serially diluted HIV-1 RNA samples;

FIG. 49 shows average velocity of motors (n=30) with and without HIV-1 LAMP amplicons generated from HIV-1 RNA concentration of 10⁴ copies/μL;

FIG. 50 shows the average velocity of motors in the presence of 0% to 100% dilutions of HIV-1 LAMP amplification products prepared in LAMP reaction buffer;

FIG. 51 shows agarose gel electrophoresis image of HIV-1 and human papillomavirus 16 (HPV-16) and different non-targeted viruses;

FIG. 52 shows the average velocity of motors in the presence of the amplification products on the target and non-target viruses;

FIG. 53 shows representative digital images showing the motion trajectories of motors in the presence of LAMP amplification products generated with the target and non-target viruses;

FIG. 54 shows a bar graph showing the average velocity of motors recorded by the CALM system for phosphate-buffered saline (1×PBS, pH 7.4) samples (n=45) spiked with different HIV-1 RNA concentrations;

FIG. 55 shows representative digital images showing the motion trajectories of motors in the absence of HIV-1 RNA or the presence of HIV-1 RNA at concentrations above and below the threshold of 1000 copies/mL;

FIG. 56 shows a heatmap of the average motor velocity measured by the CALM system for different virus concentrations spiked in the 1×PBS (n=35) and serum (n=20);

FIG. 57 shows receiver operating characteristics (ROC) curve analysis of 1×PBS (n=35) and serum (n=20) samples spiked with different HIV-1 concentrations showing assay detection sensitivity (sens) and specificity (spec) compared to real-time polymerase chain reaction (RT-PCR);

FIG. 58 shows a vertical scatterplot analysis of virus spiked samples (n=54); and

FIG. 59 shows representative digital images of motion trajectories of motors in the absence of HIV-1 particle and the presence of HIV-1 at concentrations above and below 1000 virus particles/mL.

### DETAILED DESCRIPTION

### I. Definitions

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups. As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “point-of-care diagnostic” can refer to test can be performed at the time and place of sample collection. Results of the test can also be read at the time and place of sample collection.

As used herein, the term “sample” can refer to a small part or quantity intended to show what the whole is like. The sample can be, for example, a biological sample, a chemical sample, an environmental sample, or the like.

As used herein, the term “analyte” can refer to a substance or chemical constituent that is of interest in an analytical procedure. The point-of-care diagnostic can be related to a presence or an absence of the analyte in the sample. The term “target analyte” can be used interchangeably herein with “analyte”.

As used herein, the term “particle” can refer to a chemical substance that includes a bead and a motor structure.

As used herein, the term “motor structure” can include a catalytic motor-like structure and an attached functional material. The motor structure can be configured for self-propulsion based on a presence or an absence of an analyte.

As used herein, the term “motor-like particle” can refer to a microparticle or a nanoparticle that can employ catalytic properties to become self-propelling. The self-propulsion can be due to a conversion of chemical energy into mechanical motion (e.g., via self-electrophoresis, self-diffusiophoresis, bubble thrust, or the like) that is autonomous, powerful, remotely controlled, and/or ultrafast. Each motor-like particle can be of a spherical shape, a wire shape, a rod shape, a tube shape, a helix shape, or the like. Example materials that can be used in a motor-like particle include Au, Cu, Fe, Pd, Zn, Cd, Ag, Pt, or the like.

As used herein, the term “functional material” can refer to any type of chemical that can be specific for an analyte. The functional material can be, for example, an antibody, a nucleic acid amplicon, a DNA probe, an RNA probe, an aptamer, a protein, an intact virus, a vesicle, a cell or the like.

As used herein, the term “bead” can refer to a structure that can be optically detected (e.g., based on color, size, shape, or the like). In some instances, the bead can be modified with one or more the motor structures.

As used herein, the term “handheld device” can refer to a computing device with a processor and ability to display a visualization, such as a cellphone (e.g., a smartphone), a tablet computing device, or the like.

As used herein, the term “patient” can refer to any warm-blooded organism including, but not limited to, a human being, a pig, a rat, a mouse, a dog, a cat, a goat, a sheep, a horse, a monkey, an ape, a rabbit, a cow, etc. The terms “patient” and “subject” can be used interchangeably herein.

### II. Overview

The present disclosure relates to systems and methods that monitor particle motion behavior (related to the presence or absence of a target analyte in a sample—for example, a biological sample, a chemical sample, an environmental sample, or the like) for point-of-care diagnostics. The particle can include a bead (e.g., a microbeads) coated by one or more motor structures. Each motor structure includes a functional material specific for the analyte attached to a self-propelling catalytic motor-like structure. Accordingly, each motor structure is configured for self-propulsion based on a presence or an absence of a target analyte in the sample. One or more motor structures cause a motion behavior of a bead. The motion behavior (e.g., directed motion behavior and/or a non-directed motion behavior) of the bead can be detected and/or measured by a simple optical system (e.g., a modified cellphone or tablet computing device). The detected and/or measured motion behavior can be correlated to a diagnosis. The diagnosis can be rapid and sensitive and conducted at the point-of care.

### IIII. Systems

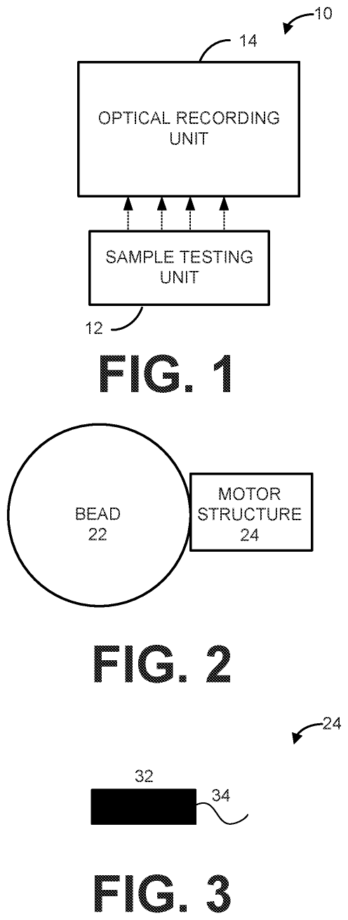

As shown in FIG. 1, one aspect of the present disclosure can include a system 10 that can monitor particle motion behavior (hydrodynamic motion), which can be caused by the presence and/or the absence of an analyte in a sample, to provide a point-of-care diagnosis. The sample can be a biological sample, a chemical sample, an environmental sample, or the like. As an example, the sample can be blood, sweat, urine, or other type of biological fluid and the analyte can be related to a pathogen (like a bacteria, a virus, a fungus, etc.), a cancer indicator, a generic marker, or the like.

The system 10 can include a sample testing unit 12 and an optical recording unit 14. At least a portion of the sample testing unit 12 can be configured to house the sample and a plurality of beads that experience a detectable motion behavior in the presence of the analyte. The optical recording unit 14 can be configured to detect the motion behavior of the beads in the sample testing unit 12. Notably, the motion behavior can be indicative of the presence or the absence of the analyte.

The optical recording unit 14 includes an optical arrangement that can be configured to detect the motion behavior of the beads. In some instances, the optical arrangement can include a handheld device that can include a processor and be configured to record images and/or video of the sample to detect the motion behavior. For example, the handheld device could be in the form of a cellphone (e.g., a smartphone), a tablet computing device, or the like. In some instances, the optical arrangement can also include an imaging adjustment configured to magnify any images taken. For example, the images can be optical images taken with a standard camera that comes with the handheld device; the imaging adjustment can be placed over the camera to provide magnification, color emphasis, or the like. The sample testing unit 12 can be placed relative to the optical recording unit 14 so that an image can be taken of at least a portion of the sample. The sample testing unit 12, in some instances, can include a device (e.g., a microchip with at least one channel that can be loaded with the sample or the like) that can hold the sample and fit within an attachment for the handheld device. At least a portion of the device holding the sample can be imaged by the optical recording unit 14. In some instances, the portion of the device can facilitate or aid in the recording and/or display of the visualization to determine the presence or absence of the analyte within the sample.

As shown in FIG. 2, the sample and/or the sample testing unit 12 can include one or more beads 22 and one or more motor structure 24 associated with each of the one or more beads 22. In FIG. 2, only a single bead 22/motor structure 24 pair is shown, but it should be understood that a plurality of motor structures 24 can be associated with a single bead 22. Additionally, a sample can include a plurality of beads 22. In some instances, at least a portion of the motor structure 24 can be attached to the bead 22. However, in other instances, the motor structure 24 need not be physically attached to the bead 22. In some examples, the bead 22 can be a polymer bead (such as polystyrene or PS) with a surface that can be altered or otherwise modified to include at least a portion of the motor structure 24, creating a bead-motor structure (or bead-portion of the motor structure) complex. The bead 22 is not limited to being a polymer bead and instead may be constructed by one or more of a polymer material, a glass material, a metal material, and/or a metallic material. The beads can be detectable according to color, size, and/or shape. For example, the beads can be detectable according to their size, which is at least the size of a microbead, but may be larger than micro-size.

The motor structure 24, shown in greater detail in FIG. 3, can be configured for self-propulsion based on a presence or absence of the analyte in the sample. In its most basic form, the motor structure 24 includes a functional material 32 and a catalytic motor-like particle (such as a nanoparticle, a microparticle, or the like). The functional material 32 can be specific for the analyte (in other words, the functional material can provide a specific response to the analyte) and attached to the motor structure 24 so that the motor structure can self-propel based on the presence or absence of the analyte in the sample. The functional material 32 can include, for example, an antibody, a nucleic acid amplicon, a DNA probe, an RNA probe, an aptamer, a protein, an intact virus, a vesicle, cell or the like (the functional material 32 may include additional stabilizing materials). The catalytic motor-like particle can include Au, Cu, Fe, Pd, Zn, Cd, Ag, Pt, or the like, and can be of a spherical shape, a wire shape, a rod shape, a tube shape, a helix shape, or the like.

As an example, the functional material 32 can provide a chemical signal in response to the analyte, and the catalytic motor-like particle 34 can convert the chemical signal into mechanical motion by at least one of self-electrophoresis, self-diffusiophoresis, bubble-thrust, or another mechanism. The motion of the catalytic motor-like particle 34 can cause the detectable motion of the bead 22. The optical recording unit 14 can detect the motion of the bead 22, which can be correlated to presence or absence of the analyte. The presence or absence of the analyte can be used to form a diagnosis.

### IV. Methods

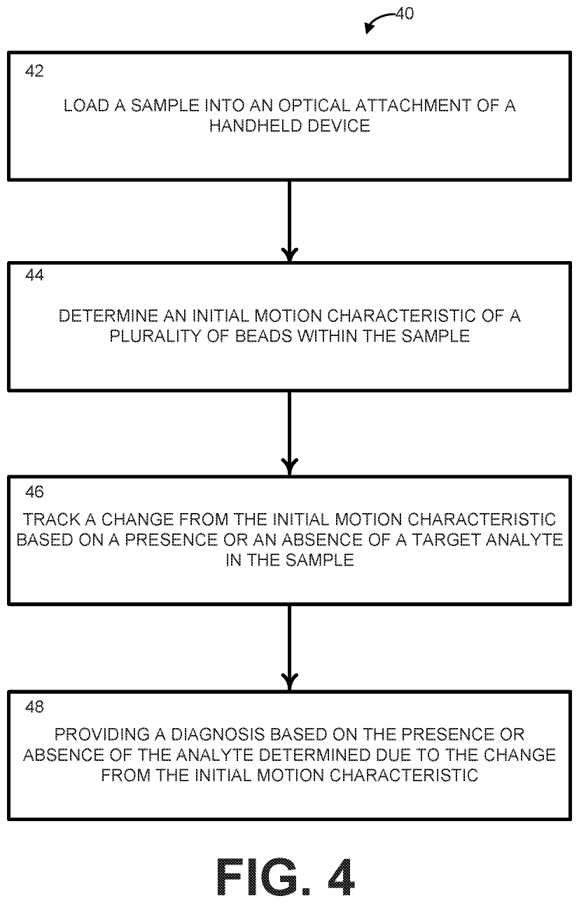

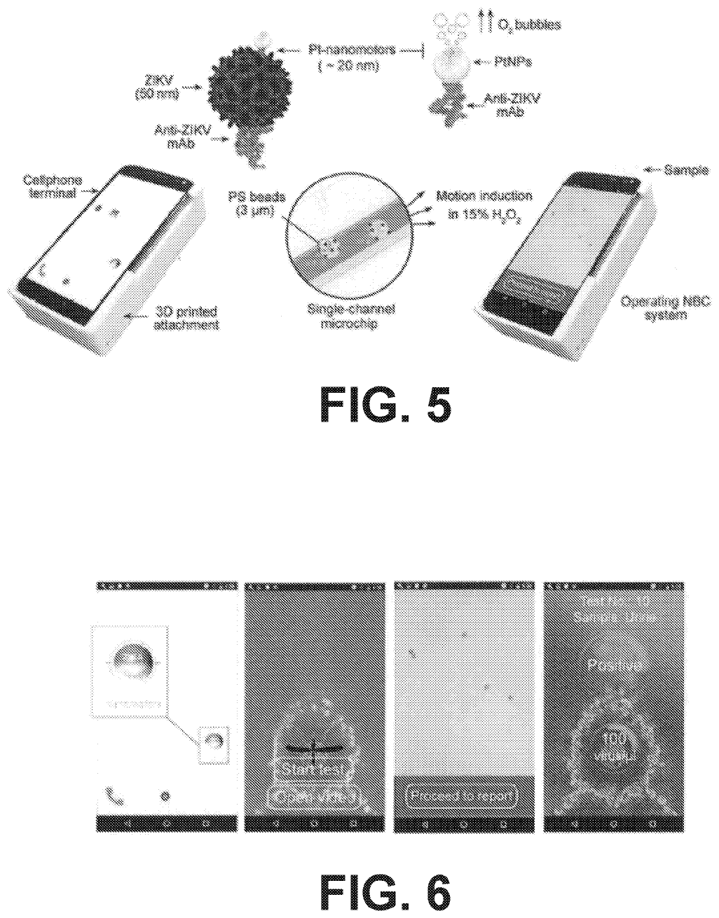

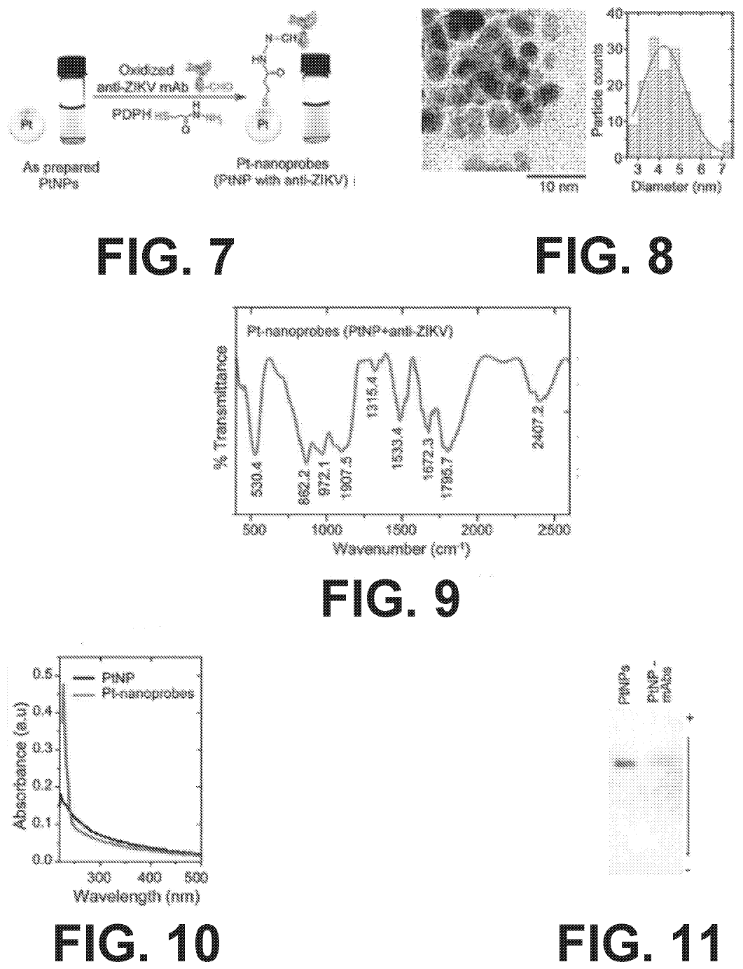

Another aspect of the present disclosure can include methods for point-of-care diagnosis based on a presence or absence of an analyte in a sample. One example of a method 40 for monitoring particle motion behavior for point-of-care diagnostics is shown in FIG. 4. The method 40 can be executed at least in part, for example, by the system 10 shown in FIG. 1.

The method 40 of FIG. 4 is illustrated as a process flow diagram with flowchart illustrations. For purposes of simplicity, the method 40 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 40.

At 42, a sample (e.g., within a portion of a sample testing unit 12) can be loaded into an optical attachment of a handheld device (e.g., the optical attachment and the handheld device can be parts of the optical recording unit 14). The sample can include a plurality of motor structures (e.g., motor structure 24) and a plurality of beads (e.g., bead 22). The plurality of motor structures can include a functional material (e.g., functional material 32) that can be attachable to a catalytic motor-like particle (e.g., catalytic motor-like micro/nanoparticle 34) to provide motion. In some instances, at least a portion of the plurality of motor structures can cause the plurality of beads to have a motion behavior (e.g., the functional material can be specific for an analyte and a reaction between the functional material and the analyte can cause the catalytic motor-like particle to cause motion, which causes an associated bead to move). For example, at least a portion of the motor structure can be attached to the respective bead to cause the bead to experience a motion behavior. When one or more motor structures (or portions of the one or more motor structures) are attached to the respective bead, a respective bead-motor structure complex is created. The bead-motor structure complex can have an attachment between the bead and the functional material and/or the bead and the catalytic motor-like particle.

At 44, an initial motion characteristic of a plurality of beads within the sample can be determined. (e.g., by the handheld device). At 46, a change from the initial motion characteristic can be tracked (e.g., by the handheld device) based on a presence or absence of a target analyte in the sample. The change can be, for example, a change in velocity, a change in direction, a change in trajectory, a change in length, and/or any other change related to the motion characteristic. In some instances, the handheld device can display a visualization of the beads to show the initial motion characteristic and track the change from the initial motion characteristic over time.

At 48, a diagnosis can be provided (e.g., at least in part by the handheld device) based on the presence or absence of the analyte determined due to the change from the initial motion characteristic. As an example, the handheld device can provide a report related to the target analyte (e.g., including the presence of the target analyte, a concentration of the target analyte, a concentration range of the target analyte, etc.) and a medical professional who reads the report can finalize the diagnosis. As another example, the handheld device may offer a proposed diagnosis that can be approved by the medical professional who reads the report. This step is not strictly necessary because the medical professional may make the diagnosis based on the change alone.

### V. Examples

The following examples are for the purpose of illustration only and is not intended to limit the scope of the appended claims. Example 1 relates to detection of the Zika virus (“ZIKV”). Example 2 relates to the detection of Human Immunodeficiency Virus (HIV-1).

### Example 1—Zika Virus (“ZIKV”)

Zika virus (“ZIKV”) is spread by the bite of an infected mosquito and can be passed from a pregnant woman to her fetus, causing certain birth defects, including microcephaly and other neurological complications like Guillain-Barre syndrome. Since no preventative vaccine or specific medication exists for ZIKV, sensitive and rapid diagnosis of ZIKV has become a critical and urgent public health demand. This Example demonstrates the development of a system for the sensitive and rapid diagnosis of ZIKV. The system can detect ZIKV by leveraging the catalytic properties of Pt-nanomotors that were prepared with Pt-nanoparticles (PtNPs) modified with antibodies to induce the motion of microbeads in the presence of ZIKV under a cellphone optical system (FIG. 5). However, the system used in this Example can be extrapolated to include the motion-based detection of other analytes, including other target viruses, using the catalytic activity of nanomotors constructed using different antibodies.

Methods

### Virus Culture and Isolation

Zika virus PRVABC59 isolated by the U.S. Center for Disease Control (CDC) from a ZIKV-infected patient who traveled to Puerto Rico in 2015 (NCBI accession no. KU501215) was used in this study. Virus stock was received from CDC and propagated in the Vero cell line c6/36 following standard protocols. Cells were grown until confluence was reached. Then the growth medium was discarded, and fresh media was added and warmed up to 33° C. Virus was then added to the cells and incubated at 5° angle for 1 h in the incubator at 33° C. DMEM-5 was again added and incubated for 6 days at a slant angle of 20° in an incubator at 33° C. The virus was harvested by centrifuging the cell culture media at 4000×g for 30 min at 4° C. The supernatant was then collected and aliquoted into separate vials containing 500 μL each.

### Virus Purification and Quantification

Zika virus particles were purified by centrifugation on sucrose gradients. 24 mL of virus supernatant was loaded into an ultracentrifuge tube, and 7 mL of 20% sucrose solution was slowly added to the bottom of the tube. The tubes were then centrifuged for 3.5 h at 100,000×g and 4° C. Then the formed virus pellet dried upside-down inside the biosafety cabinet at room temperature for 20 min. The virus was suspended in DMEM-30 and quantified by RT-PCR using a Zika Real Time RT-PCR Kit (MyBiosource, Inc., San Diego, Calif., USA).

### Microchip Fabrication

The microfluidic device consists of three layers: PMMA (3.175 mm; McMaster-Carr, 8560K239) that contains the inlets and outlets of microchannels, double-sided adhesive (DSA) sheet (80 mm; 3M, 82603) that includes a single microfluidic channel, and a glass slide (25×75 mm; Globe Scientific, N.J., USA). The microchip design was initially prepared using the vector graphics editor CorelDraw X7 software. Then, the DSA and PMMA were cut using the VLS 2.30 CO₂ laser cutter (Universal Laser systems AZ) with the laser power, speed, and pulse per inch of 93%, 2.3%, and 1000, respectively, for PMMA and 20%, 15%, 500, respectively, for DSA. All the materials used in the microchip preparation, including PMMA, DSA, and glass slides, were cleaned with ethanol, H₂O₂, and DI water using lint-free tissues. The DSA was then peeled off of one side and was applied to the clean side of the PMMA. After ensuring that the DSA was added properly, the other side of the DSA was peeled off and was stuck on to the precleaned glass slide.

### Nanomotor Preparation and Characterization

Platinum nanomotors that specifically recognize ZIKV were prepared of spherical platinum nanoparticles (PtNPs) modified with monoclonal anti-Zika virus (ZIKV-Env) antibody (EastCoast Bio, Inc. North Berwick, Me., USA, cat no. HM325). The synthesis protocol begins with PtNPs synthesis followed by antibody coupling to the surface of the PtNPs. PtNPs were synthesized using a modified method from literature. All glassware used was cleaned with aqua regia and ultrapure water. 36 mL of a 0.2% solution of chloroplatinic acid hexahydrate was mixed with 464 mL of boiling DI water. 11 mL of a solution containing 1% sodium citrate and 0.05% citric acid was added followed by a quick injection of 5.5 mL of a freshly prepared 0.08% sodium borohydrate solution, containing 1% sodium citrate and 0.05% citric acid. The reaction continued for 10 min, and the formed nanoparticles solution was gradually cooled down to room temperature. The formed PtNPs were modified with 3-(2-pyridyldithio)-propionyl hydrazide (PDPH) freshly reduced by 20 mM tris(2-carboxyethyl)phosphine (TCEP). For antibody coupling reaction, aliquots of 5 μL of antibody (7 mg/mL) were mixed with 10 mM of sodium metaperiodate and 0.1 M sodium acetate (pH 5.5) and incubated at 4° C. in the dark for 20 min. The oxidized antibody was washed by using filtration column unit (Amicon Ultra-15 Centrifugal Filter Unit, cat. no. UFC903008) and then added to PDPH activated PtNPs and allowed to react with the oxidized antibody for 1 h at room temperature. The formed Pt-nanomotors were washed by a dialysis membrane using phosphate buffer for 3 h with mild stirring at 4° C. The prepared PtNPs and Pt-nanomotors were characterized using transmission electron microscopy (TEM), ultraviolet-visible (UV-vis) spectroscopy, Fourier transform-infrared spectroscopy (FT-IR), potential, and dynamic light scattering (DLS).

### Bead Modification and Characterization

ZIKV was captured on the surface of 3 μm PS beads and labeled with Pt-nanomotors. The protocol used for this step involves three main reactions: (1) Polystyrene beads activation with adipic acid. In this reaction, 20 μL of Sperotech-SPHERO carboxyl beads with 1% w/v was diluted in 200 μL of 0.05 M 2-(N-morpholino)ethanesulfonic acid (MES) pH 5.0, then activated using EDC-NHS coupling reaction by adding 100× molar concentration of adipic acid dihydrazide. The reaction mixture was incubated at room temperature with agitation for 20 min. After the reaction, the activated beads were washed twice with MES buffer. (2) Anti-ZIKV monoclonal envelope antibody oxidation using sodium periodate following the described protocol in the previous section. (3) Oxidized coupling to the surface of hydrazide beads. The surface area of beads and the concentration of antibody was calculated and adjusted in a way that it covers 20%, 40%, 80%, and 100% of the beads. After optimization, the ratio of antibody covering the beads was optimized to be 40%. Antibody was added to the activated beads and was incubated for 2.5 h on the shaker 150 rpm. Excess antibody was washed twice. PBS was used as storage buffer for the modified beads and kept in dark at 4° C. The prepared beads modified with anti-ZIKV antibody were characterized using UV-vis spectroscopy and FT-IR techniques.

### Bead Motion Cellphone Assay

The NBC system assay relies on the induction of the bead motion in the presence of target virus due to the formed bead-virus-PtNP complex. The working protocol comprises three main steps: (1) Virus capture on the surface of beads. 5 μL of the antibody-modified beads were added to a 1.5 mL centrifuge tube, 10 μL of ZIKV was added, and the final volume was made up to 100 μL with 100 mM phosphate buffer (pH 7.2). The sample was incubated for 20 min with mild shaking (150 rpm) at room temperature and washed twice with phosphate buffer to remove all non-captured viruses from the sample. (2) Pt-beads-virus complex formation. 20 μL of prepared Pt-nanomotors were added to the centrifuge tube and incubated for 20 min with mild mixing. The sample was washed 3 times using phosphate buffer to remove all free nanomotors. (3) Motion testing using the cellphone system. H₂O₂ solution (30%) was mixed with equal volume of the prepared Pt-bead-virus complex solution. 10 μL of the mixture was loaded on the microfluidic device, and the motion of the beads was measured using the developed cellphone system. The capture of ZIKV on the surface of beads was confirmed using SDS gel electrophoresis and SEM techniques. The induction of the beads motion in the presence of ZIKV was initially tested using bright-field light microscopy technique. Videos of virus-free control and ZIKV-spiked samples (10⁶ particles/μL) were recorded under light microscopy using Snagit at 10 frames per second. Then videos were analyzed using ImageJ and MtrackJ plug-in to calculate the velocities of beads.

### Detection and Performance of the NBC System

The sensitivity of the NBC system was evaluated using serially diluted ZIKV-spiked PB samples with virus concentrations ranging from 10° particles/μL to 10⁶ particles/μL. 10 μL of each virus concentration was tested using a bead motion testing protocol, and 10 μL of the formed reaction mixture was loaded into the microchip and were immediately tested with the cellphone. This process was repeated for all of the samples with different virus concentrations. One positive control with ZIKV and without nanomotors was included in all of the three trials. The specificity of the developed NBC assay was tested using ZIKV and non-target viruses, including DENV-1, DENV-2, CMV, and HSV-1 at 10⁶ particles/μL using the same protocol.

### Cellphone Optical System and Software

The cellphone setup was designed using Solid Works 2015 software and 3D printed with a 3D printer (Ultimaker Extended II) using Ultimaker PLA (polylactic acid) as printing material. The setup was designed to record the videos S70 using the cellphone rear camera. The optical cellphone attachment has an LED, electronics and switches, and lenses for image magnification. The electronic switch on the optical system is used to turn on and off the light source when needed. A Moto X smartphone (Motorola, XT1575) was used in this work. A microchip holder was engraved on the cellphone optical attachment for microchip manipulation and positioning. The cellphone application was designed using Android Studio. The cellphone application records a video of the sample for 2 min at 30 frames per second. The detection algorithm identifies the beads and tracks their motion to calculate the velocities. The virus concentration is calculated based on the bead motion change in the sample. The cellphone application is enabled with a user-friendly interface that can be operated by a lay user.

### System Evaluation Using Spiked and ZIKV-Infected Patient Samples

To evaluate the NBC system, ZIKV-spiked synthetic urine and artificial saliva samples were used with virus concentrations of 10¹ particles/μL, 10³ particles/μL, and 10⁵ particles/μL. ZIKV-infected serum patient samples (n=10) purchased from Boca Biolistics, LLC (Pompano Beach, Fla., USA) were also used for system evaluation. Each spiked sample was tested using our bead motion testing protocol for performance testing of NBC system.

### Statistical Analysis

Statistical analyses were performed using OriginPro 2015 (OriginLab Corporation, Northampton, USA) and GraphPad Prism software version 5.01 (GraphPad Software, Inc. La Jolla, Calif., USA). Data were collected and analyzed using software, and each data point represents the average of a total of three independent measurements.

Results

### NBC System Design and Development

In the assay shown in FIGS. 5 and 6, the applied Pt-nanomotors were specifically designed to interact with ZIKV captured on the surface of 3 μm polystyrene (PS) beads, forming a three-dimensional (3D) immunocomplex that moves in the presence of H₂0₂. While loaded on the surface of a single-channel microchip, the average motion velocity of the formed immunocomplexes (beads-virus-motors) is measured by a cellphone enabled with an optical attachment and a motion tracking cellphone application. The average motion velocity of the beads was then quantitatively correlated to the virus concentration in the tested sample. The Pt-nanomotors were mainly comprised of PtNPs conjugated with anti-Zika virus monoclonal antibody (anti-ZIKV mAb) specifically targeting the envelope protein. The motors move by catalyzing the decomposition of H₂0₂, and thus in the presence of ZIKV, an abundant number of motors accumulates on the surface of the beads and induces their motion. In contrast, in the absence of virus, the motors did not bind to the surface of beads and remain free in H₂O₂ solution, resulting in a significantly lower motion velocity of beads as compared to when target viruses were present in the sample. The cellphone setup used in this study comprises an android terminal modified with a cellphone application, a disposable microchip, and an optical cellphone attachment. A MotoX cellphone (Motorola, ZT 1575) was used in performing the experiments in this work. The optical cellphone attachment was designed using SolidWorks 2016 software and fabricated using a 3D printer (Ultimakerll Extended) with Ultimaker PLA (polylactic acid) as printing material. The microchip was prepared with two main layers of glass slide and poly(methyl methacrylate) (PMMA) that were assembled together using a laser-machined double-sided adhesive (DSA) sheet to form a single longitudinal channel. The optical attachment includes an inexpensive acrylic lens for image magnification, electronics, and a LED light source. A slide holder was engraved on the cellphone attachment where the microchip can be inserted into the setup and imaged. A customized cellphone application was developed to specifically identify beads in the sample and track its movement to measure its velocity and calculate the virus concentration. The cellphone application can record videos, enumerate beads, automatically calculate their motion velocity, and report the results in ˜2 min. The cellphone application is enabled with a user-friendly interface to facilitate the testing process. The developed system was able to record videos of a sample at a rate of 30 frames per second (fps) with a maximum effective field-of-view (POV) of 480×360 μm. The device was calibrated using a micrometer scale. The resolving power of the attachment was tested using micropolystyrene beads (3 μm). It was observed that the system was able to visualize and detect the motion of the microbeads.

### Pt-Nanomotors Preparation and Characterization

Pt-nanomotors were prepared from PtNPs functionalized with anti-ZIKV mAb following a surface chemistry protocol that relies on using a bifunctional cross-linker of 3-(2-pyridyldithio)propionyl hydrazide (PDPH) to bind the oxidized antibodies through their carbohydrate residues to the surface of nanoparticles (FIG. 7). Transmission electron microscopy (TEM) and the corresponding size distribution histogram indicate that the synthesized PtNPs are spherical in shape with an average diameter of 4.37±0.986 nm (FIG. 8). Fourier transform-infrared spectroscopy (FT-IR) was performed to characterize the surface chemistry and antibody immobilization. The conjugation of mAb to PtNPs resulted in several peaks in FT-IR analysis that are characteristic for antibodies. FIG. 9 shows FT-IR spectra of Pt-nanomotors with different bands appearing at 2407.2, 1672.3, 1533.4, 1315.4, 1907.5, and 862.2 cm⁻¹, which can be assigned to C═O stretching, N—H bending, C—N stretching, C—C stretching, and S-metal bond, respectively. These bands correspond to the thiol-Pt bond formed by PDPH with the surface of PtNPs and to amid-I and -II characteristic of antibodies coupled to the surface of the PtNPs. Ultraviolet-visible (UV-vis) analysis of citrate-stabilized PtNPs and Pt-nanomotors (PtNPs modified with mAb) confirmed the stability of the synthesized nanomotors, and a strong absorption peak was observed at 223 nm, which is associated with the presence of the antibody as a protein structure (FIG. 10). On the other hand, the conjugation of antibodies to the surface of PtNPs caused retardation in the motion of the formed Pt-nanomotors (PtNPs-mAbs) compared to non-modified PtNPs when tested on agarose gel electrophoresis, which can be attributed to the difference in size and charge density value between PtNPs (no antibodies) and the formed nanomotors (PtNPs-antibody conjugates) (FIG. 11). The ratio of antibody molecules per nanoparticle was estimated to be 1.792±0.693 antibody molecule/PtNP based on their corresponding absorption values at 223 nm. Therefore, approximately 6.38% of the surface of Pt-nanomotor particle was covered with anti-ZIKV mAb, and 93.62% of the surface of PtNPs was available to interact with H₂O₂ for gas formation. In addition, this ratio of antibody surface coverage on Pt-nanomotors allows efficient labeling of the captured virus with minimum chance for the formation of large aggregates of beads, which can limit the motion of each complex and result in a false negative signal.

### Preparation and Characterization of ZIKV Capturing Beads

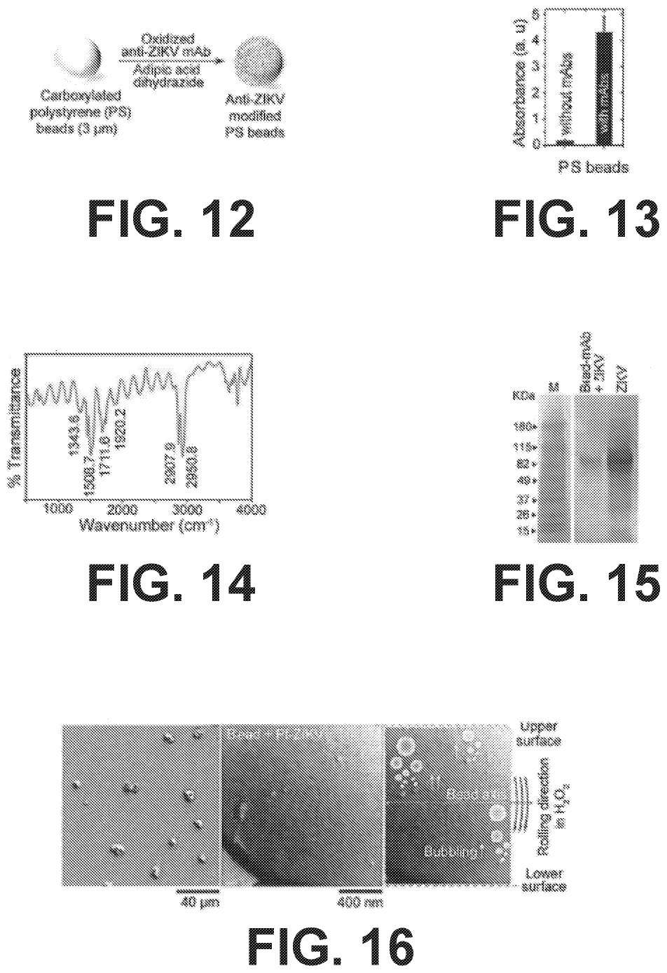

Beads coated with anti-ZIKV envelope mAb were used to allow specific formation of Pt-bead virus complexes by the accumulation of nanomotors on the surface of beads in the presence of ZIKV. Beads conjugated with anti-ZIKV mAb were prepared using a coupling protocol that allows the directional conjugation of antibodies to the surface of beads using adipic dihydrazide (FIG. 12). Carboxylated beads were initially activated with adipic acid using the well-known-I-ethyl-3-(3-(dimethylamino)propyl)carbodiimide hydrochloride (EDC)/sulfo-N-hydroxysuccinimide (sulfa-NHS) protocol. Then the free hydrazide groups on the surface of the beads were directly coupled to oxidized antibodies by sodium periodate and through their carbohydrate residue in FC region. The surface activation of beads with adipic acid was confirmed using ζ potential and FT-IR techniques. The ζ potential indicated a significant decrease to −10.2±2.21 mV in the net negative surface charge of the beads after the activation with dihydrazide due to the presence of terminal amine with positive charge FT-IR indicated the presence of 1720.5 and 1666.5 cm⁻¹ vibrations that are characteristic for amide-I and -II groups of the adipic dihrydrazide existing on the surface of beads. For antibody conjugation1 UV-vis spectroscopy indicated the presence of a strong peak at 223 nm that is specific for the antibody used in this study. Using a standard curve prepared of different concentrations of anti-ZIKV antibody, the number of antibody molecules per each bead was ˜3×10⁴ molecule (FIG. 13). On the other hand1 FT-IR analysis of bead-modified with antibodies showed the presence of a cluster of vibration bands at 1343.6, 1508.7, 1711.6, and 1920.2 cm⁻¹ and two bands at 2907.9 and 2950.8 cm⁻¹. The peaks at 1343.6, 1508.7, 1711.6, and 1920.2 cm⁻¹ can be attributed to the amide-I and II characteristic to the antibody as protein structures, while the peaks at 2907.9 and 2950.8 cm⁻¹ can be attributed to C═O of the carboxyl m groups in the PDPH cross-linker used in antibody conjugation m protocols (FIG. 14) Virus capture and PtNP-virus complex formation on the surface of beads were confirmed using sodium dodecyl sulfate polyacrylamide (SOS) gel electrophoresis, scanning electron microscopy (SEM), and inductively coupled plasma mass spectroscopy (ICP-MS) techniques. SOS gel electrophoresis analysis of the virus captured on beads indicated the presence of an intense band at 88.2 kDa that is characteristic to the non-structural protein 3 (NS3) of ZIKV and also appeared in the control sample of purified ZIKV in PBS (FIG. 15). SEM analysis of PtNP-virus assemblies on the surface of beads indicated that the formed assemblies were 80-100 nm in size and that there were approximately 1.2 PtNP-virus complexes per 1 μm² (FIG. 16). The formed PtNP-virus-beads complexes were isolated by centrifugation, washed, and tested by ICP-MS technique to estimate the concentration of Pt metal on the surface of beads. The results confirmed the accumulation of Pt-nanomotors on the surface of beads in the presence of ZIKV at the rate of 1200 Pt-nanomotors per bead when 10³ virus particles/μL were used in the reaction.

### Validation of the NBC System Design for ZIKV Detection

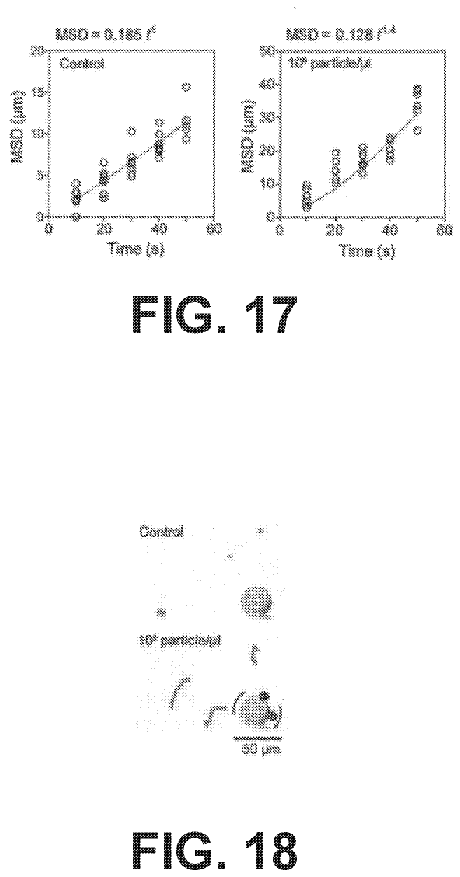

The motion induction of PS beads initially was tested in the presence of virus particles using bright-field microscopy. Aliquots of ZIKV-spiked phosphate buffer (PB) with virus concentrations of 10⁶ particles/μL were added to antibody-modified beads followed by the addition of Pt-nanomotors to allow the formation of PtNP-bead-virus complexes. The motion of the formed complexes was then tested in 10% H₂O₂ solution in a single channel microfluidic chip under light microscope and using ImageJ software. FIGS. 17 and 18 shows the difference between the motion of beads in the presence and absence of the target ZIKV. Using mean squared displacement (MSD) versus time t as a model to assess the motion of beads, the MSD versus time had a near linear dependence (α=1.0), which is typical to the case for random diffusion where α=1 in virus-free control samples. On the other hand, when 10⁶ virus particles/μL was used in 10% H₂O₂, a power dependence with α=1.4 was observed indicating that the bead motion is specifically correlated to the presence of ZIKV (FIG. 17). The beads in ZIKV-spiked samples were observed to move with an average velocity of 1.199 μm/sec, which was approximately 5.2-times higher than the average velocity of the beads in virus-free control samples. These results confirmed the ability of the virus to specifically induce the motion of beads when the bead-virus-PtNP complexes were formed. Trajectory images of the motions of beads in control ZIKV-spiked samples are shown in FIG. 18. Furthermore, the change in the velocity of bead motion was tested upon induction with different concentrations of ZIKV (from 0 particles/μL to 10⁵ particles/μL). The results confirmed the ability of the virus to induce the motion of beads when assembled with Pt-nanomotors in 15% H₂O₂ solution. The velocity of bead motion increased significantly with the increase in virus concentrations tested. The ability of the developed NBC system to track the motion and measure the velocity of beads was then validated using samples with different concentrations of ZIKV (n=40). The motion tracking cellphone application was optimized to have a correlation percentage of 89.11% with the measurements obtained by light microscopy and ImageJ software.

### Evaluation of the NBC System in ZIKV Detection Using Spiked Patient Samples

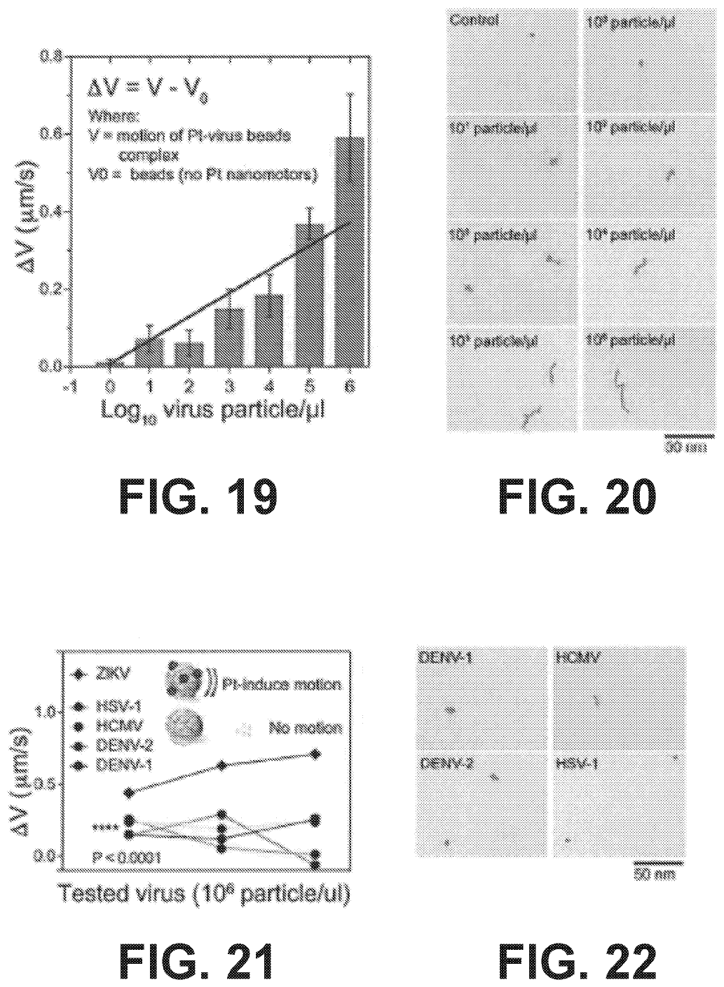

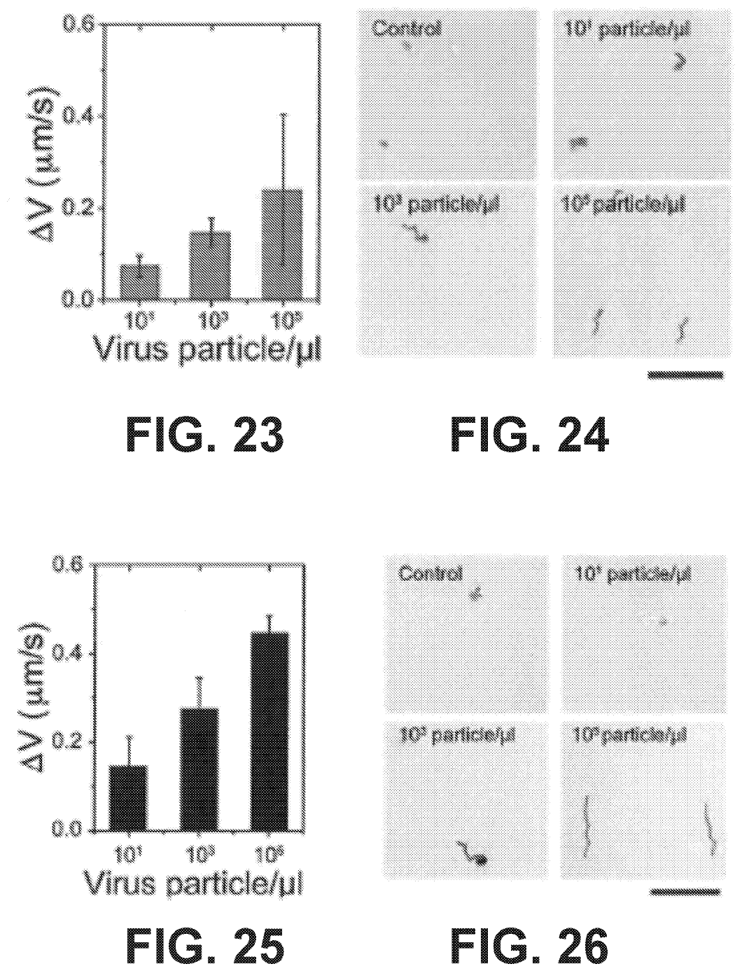

To evaluate the performance of the NBC assay in Zika detection, samples spiked with different ZIKV concentrations ranging from 10⁰ particles/μL to 10⁶ particles/μL and non-target viruses, including dengue types 1 (DENV-1) and 2 (DENV-2), human simplex virus type 1 (HSV-1), and cytomegalovirus (CMV) were used. The antibody-modified beads were mixed with the samples for virus capture on beads and then incubated with Pt nanomotors to allow the formation of beadvirus-NPs complexes. The motion of the beads was then monitored in 15% H₂O₂ solution using the cellphone optical system. FIG. 19 shows the change in the beads motion velocity (A V) caused by the addition of different concentrations of ZIKV. The results showed an excellent correlation (r²=0.89) between the virus concentration and the magnitude of the beads velocity change. Based on S/N=2.0 and S/N=3.0, the detection limit of the developed NBC assay for testing the target ZIKV in PB is down to 1 particle/μL and 10 particles/μL, respectively. Trajectory images of the motions of beads with different ZIKV concentrations as recorded by the NBC system are shown in FIG. 20. In addition, the detection specificity of the system was tested using the same concentration (10⁶ particles/μL) of the target ZIKV and non-target viruses including DENV-1, DENV-2, HSV-1, and CMV. FIG. 21 presents the change in beads velocity (A V) with ZIKV, DENV-1, DENV-2, HSV-1, and CMV. The increase in the beads velocity caused by the target ZIKV was significant (P<0.0001) compared with the change in the velocity of beads caused by the addition of all the non-target viruses. FIG. 22 shows representative digital images of the trajectories of beads in the presence of DENV-1, DENV-2, HSV-1, and CMV. The images indicate a relatively slow motion of beads that is comparable to the random diffusion of beads in control samples (FIG. 20). To further confirm the potential of the NBC system for virus detection in biological samples, the developed system was tested using urine and saliva samples spiked with ZIKV (n=9) at three different concentrations (i.e., 10¹ particles/μL, 10³ particles/μL. and 10⁵ particles/μL) (FIGS. 23-26). FIGS. 23 and 24 show that the beads velocity change increased as the virus concentration in the sample increased and the cellphone system was able to detect ZIKV in urine samples at concentrations as low as 10¹ particles/μL. There was a variation in the motion velocity recorded for urine samples with relatively high virus concentration (10⁵ particles/μL), which can be explained by the variation in the number of captured virus particles to different beads in the presence of high salt concentration and unfavorable pH conditions. However, significant beads velocity change was not observed in virus-free control samples. These results confirmed the ability of the NBC system for target virus detection and particularly Zika virus in complex biological samples such as urine and saliva. Furthermore, we evaluated the performance of the NBC system in identifying the ZIKV-infected serum samples compared to the results obtained by the standard techniques currently approved by the U.S. Food and Drug Administration (FDA) and recommended by the Center for Disease Control (CDC) for qualitative detection of ZIKV, including CDC Zika MACELISA (ZIKV IgM detection) and Aptima Zika virus assay (ZIKV RNA detection) (Table 1). The results indicated that the accuracy of the NBC system in classifying patient serum samples as positive (ZIKV-infected) and negative (non-infected) compared to CDC Zika MAC-ELISA was 100%, while having a correlation of 80% to Aptima Zika virus assay.

TABLE 1
NBC System Evaluation using ZIKV-Infected
Patient Serum Samples (n = 10)

| | CDC Zika | | Aptima Zika virus | | | |
|---|---|---|---|---|---|---|
| | MAC-ELISA^{b} | | assay^{c} | | NBC system^{d} | |
| patient | | ISR | | S/Co | | V |
| no. | sample | value | sample | value | sample | (μm/s) |
| 1 | positive | 13.86 | positive | 18.51 | positive | 0.272 |
| 2 | positive | 14.96 | negative | 0.00 | positive | 0.253 |
| 3 | positive | 21.32 | positive | 31.91 | positive | 0.432 |
| 4 | positive | 3.78 | positive | 20.59 | positive | 0.208 |
| 5 | positive | 18.98 | positive | 32.91 | positive | 0.439 |
| 6 | positive | 17.25 | negative | 0.00 | positive | 0.372 |
| 7 | positive | 18.07 | positive | 33.09 | positive | 0.475 |
| 8 | positive | 14.14 | positive | 19.39 | positive | 0.246 |
| 9 | positive | 2.55 | positive | 17.18 | positive | 0.140 |
| 10 | negative | 0.60 | negative | 0.00 | negative | 0.065 |

The development of the NBC system for sensitive and specific detection of ZIKV by leveraging the advantage of catalytic properties of Pt-nanomotors that were prepared with PtNPs modified with antibodies to induce the motion of microbeads in the presence of target virus was demonstrated. This study integrates bead motions and cellphone for the detection of viruses by using specifically designed Pt-nanomotors. The high sensitivity (1 particles/μL, S/N=2) of the NBC system is attributed to the efficient catalytic activity known for the PtNPs used in the preparation of Pt-nanomotors in this study. PtNPs with ˜4.4 nm in diameter were specifically used in the preparation of nanomotors to allow maximum accumulation of nanoparticles on the surface of virus particles captured on the beads, which led to efficient induction of the motion of beads even at low concentrations of viruses. The ratio of anti-ZIKV monoclonal antibody was controlled at ˜1.8 antibody molecules per nanomotor to preserve the catalytic activity of the motors without affecting their efficiency to interact with captured viruses on the surface of the beads. This optimum antibody concentration per PtNP further prevents the formation of aggregates during assay. Due to the limitation in visualizing nanomotors (<1000 nm in all dimensions) using cellphones even with advanced optics, beads that are micrometer in size are used in the NBC system to allow visualization of the motion change using a low-cost cellphone-based optical sensor. In the NBS system, 3 μm PS beads with a density of 1.1 g/cm were used to minimize the effect of gravity forces on the beads and to increase the efficient detection time. Large beads can be easily observed using a cellphone with the aid of simple optical accessories. However, on the negative side, larger beads can experience larger hydrodynamic resistance in the solution, which demands a higher amount of nanomotors to cause a significant and detectable bead motion change. Also, it was necessary to use highly uniform beads that were within ˜0.16 μm variation in size to avoid any effect on the velocity of beads because of size variation. It is worth mentioning that the use of microparticles allows a highly specific motion-based detection because of the absence of background signal from samples. This can further help the expansion of the system for point-of-care testing by eliminating the need for nanomotors separation or washing before motion testing with a cellphone. Furthermore, the capture of targets on beads has been known for long time to allow direct sample testing without the need for pretesting sample preparation steps, making the NBC system advantageous over the standard polymerase chain reaction (PCR)-based techniques currently recommended for ZIKV testing. Monoclonal antibodies that target the surface envelope protein and can recognize different ZIKV strains (PRVACB59, H/PF/2013, and h [symbol] 77661) were used in the preparation of both nanomotors and virus-capturing beads to allow highly specific detection of ZIKV. A combination of monoclonal and polyclonal antibodies is commonly used in capturing and labeling steps of immunoassays. Here, a monoclonal antibody was used in the preparation of nanomotors and beads to limit the formation of Pt-nanomotor bead complex in the presence of surface antigen of the virus and to improve the efficiency of the developed system for virus particle detection, which is critical for acarate detection of ZIKV infection. In addition, our antibody immobilization scheme allows a directional conjugation of antibodies to the surface of beads and nanoparticles through their FC region. The directional conjugation of antibodies helps to preserve the full activity of antibodies. It also allows highly specific interaction with the target with high avidity due to the full accessibility of Fab regions that interact with the virus on the surface of particles. The long-term shelf life and stability of the disposables used in antibody-based point-of-care diagnostics are also important. Freeze-drying the surface chemistry can prolong the stability and shelf life of the disposables. Others have demonstrated long-term shelf life for target detection on plastic chips and showed that antibodies immobilized on-chip were stable for more than 200 days.

### Example 2—Human Immunodeficiency Virus (HIV-1)

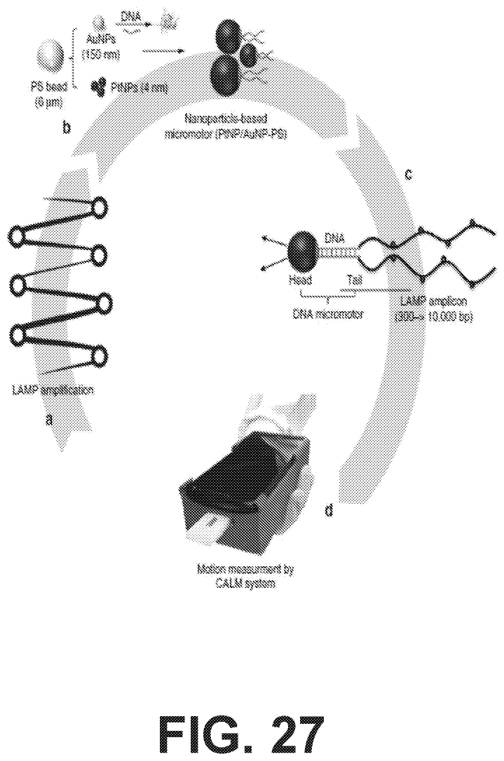

Human Immunodeficiency Virus (HIV-1) infection is a major health threat in both developed and developing countries. The integration of mobile health approaches and bioengineered catalytic motors can allow the development of sensitive and portable technologies for HIV-1 management. One such technology for HIV-1 management described herein is a platform that integrates cellphone-based optical sensing, loop-mediated isothermal amplification (LAMP), and micromotor motion (CALM) for molecular detection of HIV-1. The large stem-looped amplicons formed through LAMP amplification are uniquely adapted to change the motion of specifically DNA-engineered micromotors powered by metal nanoparticles (NPs) indicating the presence of HIV-1 using a cellphone system (example shown in FIG. 27)

Methods

### HIV-1 Propagation and Nucleic Acid Isolation

HIV-infected peripheral blood monoclonal cells (PBMCs) were first isolated from patient blood samples using Ficoll-Hypaque density gradient cell centrifugation. PBMCs were then stimulated by phytohemagglutinin and co-cultured with irradiated PBMCs at 37° C. and 5% of CO₂. The virus titer in the co-culture supernatant was tested using HIV-1 p24 antigen ELISA (PerkinElmer Life Science, Inc., NEK050b). The co-culture process was continued until the concentration of p24 became 20 ng/ml. The cell culture supernatant was collected, and the virus concentration was tested using a Roche-COBAS AmpliPrep TaqMan HIV-1 v2.0 system at Brigham and Women's Hospital (BWH). For sample testing with the cellphone system, HIV-1 RNA was isolated from each sample using the AllPrep DNA/RNA Mini Kit (Qiagen, Calif., USA) following the manufacturer's protocol.

### Microchip Fabrication

HIV-infected peripheral blood monoclonal cells (PBMCs) were first isolated from a single-channel microchip consisted of three layers: (1) PMMA (3.175 mm; McMaster-Carr Inc., 8560K239) that contained the inlets and outlets of microchannels, (2) DSA sheet (80 μm; 3M Inc., 82603) that included the microfluidic channel, and (3) glass slide (25×75 mm2; Globe Scientific Inc., 1358A). The microchip design was initially prepared using the vector graphics editor CorelDraw X7 software. Then the DSA and PMMA were machined using the VLS 2.30 CO₂ Laser cutter (Universal Laser systems AZ). The DSA was used to assemble PMMA and glass slide and the prepared chips were cleaned and tested for leakage using de-ionized water.

### Pt-Motor Preparation and Characterization

Platinum micromotors were prepared of spherical 6-μm PS beads coated with PtNPs and AuNPs and modified with DNA capture probe that recognizes HIV-1 LAMP amplicons. The detailed protocol included three main steps: (1) PtNPs and AuNPs synthesis, (2) DNA conjugation to AuNPs, and (3) PS beads surface activation and sequential coating with NPs. The synthesis of PtNPs and AuNPs was performed following the common protocol of metal salt reduction with sodium borohydride. For PtNPs synthesis, 100 ml of ultrapure water was heated in a 250-ml Erlenmeyer flask and brought to boiling and 7.2 ml of a 0.2% chloroplatinic acid hexahydrate solution was added and mixed by magnetic stirring. Then 2.2 ml of 1% sodium citrate freshly prepared in 0.05% citric acid was injected in the flask and the solution was mixed for 1 min. In all, 1.1 ml of 0.08% sodium borohydrate solution freshly prepared in 1% sodium citrate-0.05% citric acid solution was added while boiling and the reaction continued till the formation of the PtNPs. For AuNP synthesis, a seed solution of ˜15 nm-AuNPs was first prepared by adding 900 μL of 1% sodium citrate trihydrate solution to 300 μL of 1% HAuCl₄ diluted in 30 ml of H₂O. The growth reaction of AuNPs was then initiated by adding 391 μl NP seed solution to 100 μL of 1% (W/V) HAuCl₄ diluted in 9.5 ml of H₂O under rapid stirring at room temperature followed by the addition of 22 μL of 1% sodium citrate solution and 100 μL of 0.03M hydroquinone. The reduction is completed within 10 min. One milliliter of the synthesized AuNPs was mixed with freshly reduced thiolated-DNA probe deigned against HIV-1 gag gene (50 μM) and the mixture was incubated at room temperature for 12 h. The solution was then brought to 0.1M NaCl and allowed to stand for 40 h and washed twice by centrifugation at 12,000×g for 30 min using 10 mM phosphate buffer (pH 7.2). To prepare thiolated beads, 0.14 μM amine-functionalized PS beads (Spherotech, Inc., AP-60-10) were mixed with 1.6 mM SPDP crosslinker (Thermo Fisher Scientific Inc., 21857) in phosphate buffer (pH 7.2) and incubated for 3 h at room temperature. Then the thiolated beads were first coupled with the prepared DNA-AuNP conjugates using the well-known thiol-gold chemistry followed by adding excess of PtNPs to coat the remaining surface of beads. The prepared Pt-motors were characterized using TEM, UV-vis spectroscopy, FT-IR, Zeta potential (c), DLS, and ICP-MS.

### LAMP Reaction

RT-LAMP amplification of the target HIV-1 RNA was performed using a set of four specific primers (Table 2). The reaction was performed as follows: a mixture of the 4 sets of DNA primers (50 μM) was first prepared by mixing 0.8 μL of FIP, 0.8 μL of BIP, 0.1 μL of F3, and 0.1 μL of B3 and then added to the reaction mixture prepared of 2.5 μL isothermal amplification buffer (New England Biolabs Inc., BO5375), 1.5 μL MgSO4 (100 mM), 1.4 μL dNTP (25 mM), and 2.5 μL Betaine (5 M). Then 2-4 μL of the target and non-target RNA was added followed by adding of 6 unit of AMV reverse transcription enzyme (New England Biolabs Inc., M0277L) and 8-unit Bst. 2.0 DNA Polymerase (New England Biolabs Inc., M0537L). The reaction volume was brought to 25 μL by UltraPure™ DNase/RNase-Free Distilled Water (Thermo Fisher Scientific Inc., 10977023) and mixed thoroughly before incubation for 40-50 min at 65° C. and termination at 85° C. for 5 min.

TABLE 2
List of DNA sequences used.

| Oligonucleotide | Sequence |
|---|---|
| LAMP F3 primer | 5′-GGTAAGAGATCAGGCTGAACATC-3′ |
| LAMP B3 primer | 5′-GCTGGTCCTTTCCAAAGTGG-3′ |
| LAMP FIP primer | 5′-CCCCAATCCCCCCTTTTCTTAGACAG |
| | CAGTACAAATGGCA-3′ |
| LAMP BIP primer | 5′-AGTGCAGGGGGAAAGAATAGTAGACC |
| | TGCTGTCCCTGTAATAAACCC-3′ |
| Pt-motor capture | 5′-TTAAGACAGCAGTACAAATGGCAGTA |
| probe | AAAA/3ThioMC3-D/-3′ |
| Pt-motor capture | 5′-TTTTCTTTTAAAATTGTGGATGAATA |
| target DNA | CTGCCATTTGTACTGCTGTCTTAA- |

### LAMP Amplicon Capture and Motion Assay

The motion assay relies on reducing the motion of the Pt-motors when specifically coupled with the large-sized LAMP amplicons. The prepared LAMP amplicons are hybridized with Pt-motor at 80° C. for 2 min and cooled to 4° C. Then 10 μL of the formed assemblies were mixed with H₂O₂ solution and loaded on the microchip. The reduction of the bead motion in the presence of HIV-1 LAMP amplicons was tested using either the developed cellphone system or the bright-field light microscopy (Carl Zeiss AG Axio Observer D1) using Snagit v11.4.3 (Build 280) video recording software. The recorded videos were analyzed using ImageJ and MtrackJ plug-in to manually calculate the velocities of beads in the tested sample.

### Motor-Tracking Cellphone System

The cellphone attachment was designed using the Solidworks 2015 software and 3D printed using Ultimaker Extended II 3D printer and Ultimaker PLA as printing material. The cellphone attachment was designed to record the videos using the cellphone rear camera of a Moto X smartphone (Motorola, XT1575). The optical cellphone attachment has an LED, electronics, and switches and two acrylic lenses extracted from TS-H492 discarded optical drives with focal lengths of 4 and 27 mm and numerical apertures of 0.43 and 0.16. The cellphone application was designed using Android Studio to record a video of the sample for 30 s at 30 frames/s. The detection algorithm identified the motors and tracked motion of the motors to calculate average velocities. The presence of the target virus is then determined based on the change in bead motion in the tested sample.

### Evaluation of the CALM System in HIV-1 Detection

The effect of the presence of different concentrations of HIV-1 LAMP amplicons prepared by diluting the final amplification product in PB (pH 7.2) into the following percentages 100, 50, 10, 1, 0.5, 0.1, 0.01, and 0.0% was evaluated. The total DNA concentration in each dilution was first measured using a NanoDrop One-C spectrophotometer (Thermo Fisher Scientific Inc.) and 10 μL of each concentration was mixed with Pt motors and tested using the CALM system. The performance of the CALM system was evaluated using HIV-1 and non-target viruses, including HCV, HBV, HSV-1, and HPV-16. The cellphone system was calibrated with PBS samples spiked with synthetic HIV-1 RNA standard (0-1×10⁷ copies/mL) purchased from ATCC (VR-3245SD) and then compared to the standard RT-PCR using 1×PBS (pH 7.4) and serum samples spiked with HIV-1 particles at concentrations between 0 and 1.5×10⁴ virus particles/ml. In addition, the developed CALM system was tested using HIV-infected patient serum samples (n=4) and fresh whole blood from HIV-negative subjects (n=2) purchased from Research Blood Components Inc. HIV-1 plasma samples were prepared from whole blood obtained from patients enrolled in the HIV-1 Eradication and Latency (HEAL) Cohort and ART treated and followed up at BWH and Massachusetts General Hospital. This study was approved by the Partners Human Research Committee. Participants of the HEAL cohort represented a convenient sample of participants meeting the HEAL inclusion criteria. Samples obtained were based on participant flow and no other sample selection criterion was in place for the study. All patients (HIV positive and negative) provided informed consent for blood samples to be collected.

### Statistical Analysis

Statistical analyses were performed using OriginPro 2015 (OriginLab Corporation, Northampton, USA), GraphPad Prism software version 5.01 (GraphPad Software, Inc. La Jolla, Calif., USA), and MedCalc 14.8.1 (MedCalcSoftware bvba, Ostend, Belgium). Correlation between the motion tracking cellphone application and the bright-field microscopy was performed using linear regression analysis, and paired t test analysis was used to compare the motor motion analyzed by both techniques. All data for system performance were analyzed using unpaired t test analysis. Differences between groups were considered significant when P values were not >0.05, and levels of significance were assigned as *P≤0.05, **P≤0.01, ***P≤0.001, and ****P≤0.0001. Each data point represented the average of a total of three independent measurements.

Results

### Platinum-Motor Preparation and Characterization

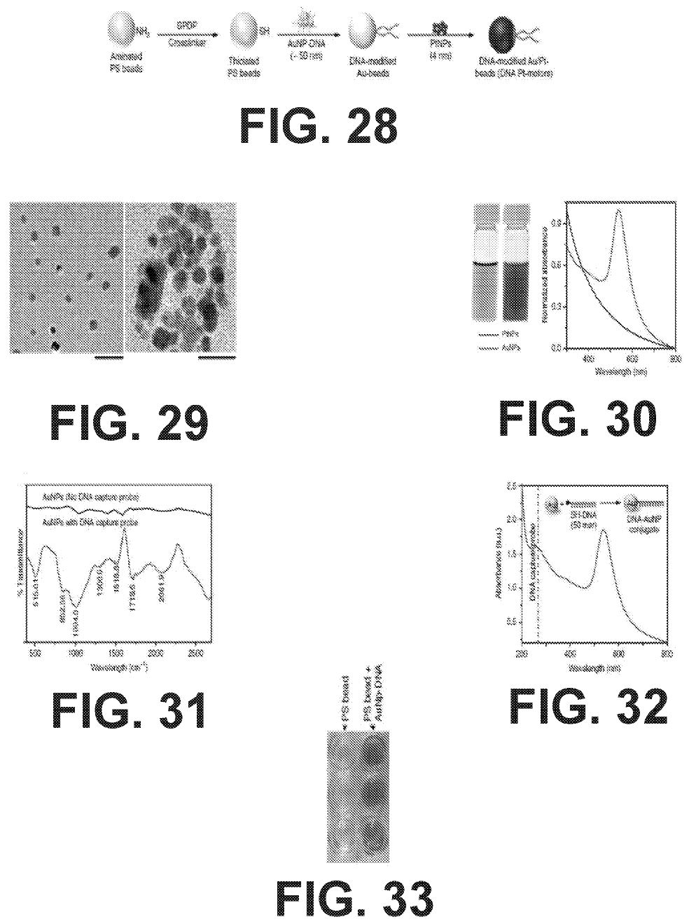

The micromotors used in this study are PtNP-coated spherical polystyrene (PS) beads (with density of 1.04 g/cm3) indirectly engineered with short DNA probes through a middle piece of spherical AuNP (FIG. 28). The motor preparation reaction includes the direct coupling of AuNPs and PtNPs to the surface of amine-functionalized PS beads using a heterobifunctional crosslinker of succinimidyl 3-(2-pyridyldithio)propionate (SPDP). The beads were initially activated with SPDP-forming thiolated beads to allow the thiol-metal-based coupling with NPs (i.e., PtNPs and AuNPs). Prior to the coupling reaction, AuNPs were modified with thiolated DNA probes of 30-mer oligonucleotides that specifically target HIV-1 gag. The prepared AuNP-DNA conjugates were mixed with the SPDP-activated beads with a molar ratio of 1:10 to minimize the number of DNA probes on the surface of beads. The remaining surface of PS beads was coated by adding excess amount of PtNPs (FIG. 28).

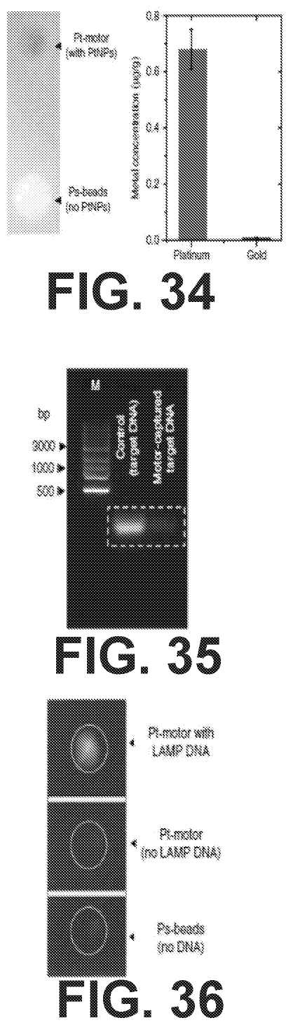

Transmission electron microscopy (TEM) of the synthesized NPs showed that both the synthesized AuNPs and PtNPs were spherical in shape with diameters of 57.721±5.181 nm (data reported as mean±standard deviation) and 3.43±1.336 nm, respectively (FIG. 29). Digital images and ultraviolet-visible (UV-vis) spectroscopic analysis results for AuNPs and PtNPs are shown in FIG. 30. In addition, dynamic light scattering (DLS) analysis results confirmed the stability of the prepared AuNPs and PtNPs with polydispersity index values <0.45 and zeta potential values of −14 to −29 mV. The conjugation of DNA to AuNPs was confirmed using UV-vis spectroscopy and Fourier transform infrared spectroscopy (FT-IR) techniques. FIG. 31 shows the FT-IR spectra of non-modified prepared AuNPs and AuNPs conjugated with DNA probes. The addition of DNA resulted into a group of peaks around 515.01, 852.56, 1004.0, 1300.0, 1518.8, 1718.6, and 2061.9/cm that are specific for NH₂ and pyridine of DNA nucleotides. In addition, the number of DNA capture probes per NP was quantified using UV-vis spectroscopy (FIG. 32). The results indicated that the average ratio of DNA/AuNPs was 12.1±0.12 DNA probe molecule per each AuNP. The efficiency of AuNP-DNA conjugates coupling to the surface of PS beads was evaluated using silver staining technique. The presence of DNA-AuNPs on the surface of beads induced a rapid change of the silver staining reaction into a dense dark brown color compared with control samples where non-modified PS beads were added (no AuNPs) (FIG. 33). The stability and reactivity of the fully structured motors (PtNP/AuNP-DNA-modified PS) were confirmed using inductive couple plasma-mass spectroscopy (ICP-MS) and gel electrophoresis techniques (FIGS. 34-36). ICP-MS analysis showed that each PS bead is modified with an average number of 1.335±0.9161 and 386,044.1±10.9161 of AuNPs and PtNPs, respectively. In addition, the deposition of PtNPs with its characteristic intense brown color on the surface of PS beads was easily observed as a visible brown color when the prepared motor solution was dropcasted on a sheet of chromatography paper, confirming the heavy surface modification of beads with PtNPs (FIG. 34). On the other hand, agarose gel electrophoresis technique was applied to test the efficiency of the fully structured motors (PtNP/AuNP-DNA-modified PS beads) in capturing synthetic target DNA (FIG. 34). Synthetic target DNA was mixed and allowed to hybridize to DNA capture probes present on the surface of Pt-motor. The hybridization reaction products of motors and target DNA were then isolated by centrifugation, washed, and the captured synthetic target DNA molecules were released by incubation at 95° C. for 5 min. The released target DNA was then tested using agarose gel electrophoresis technique. The results showed a clear band at 180 bp that is specific for the synthetic target DNA captured and isolated using the prepared Pt-motors (FIG. 35). Furthermore, fluorescence spectroscopy indicated a 30% capture efficiency of LAMP amplicons on the surface of motors (FIG. 36).

### Platinum-Motor Motion Testing and Optimization

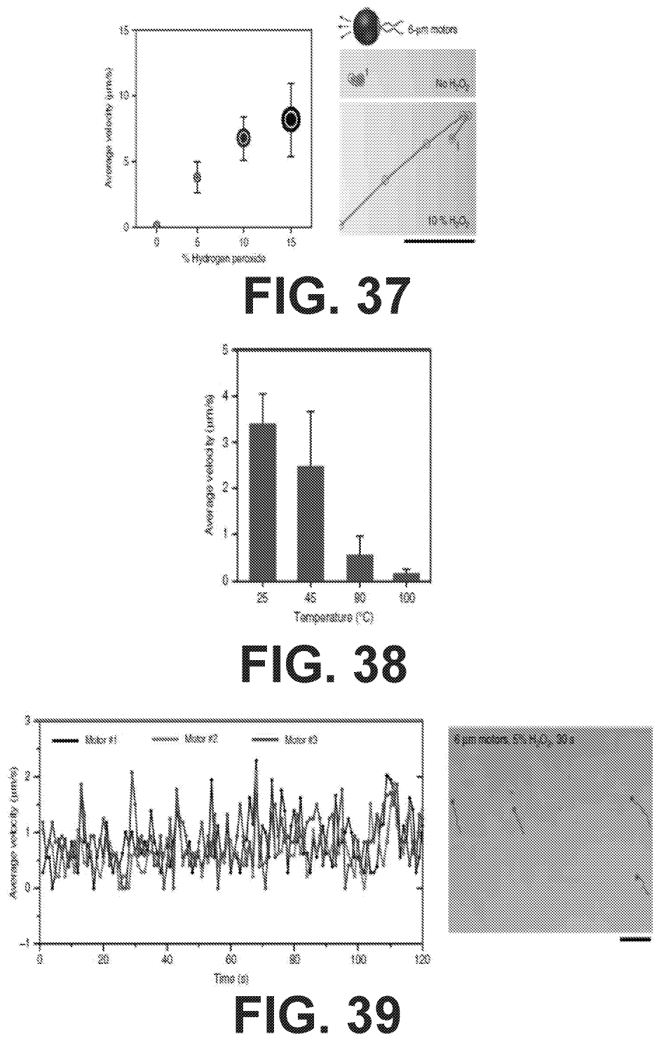

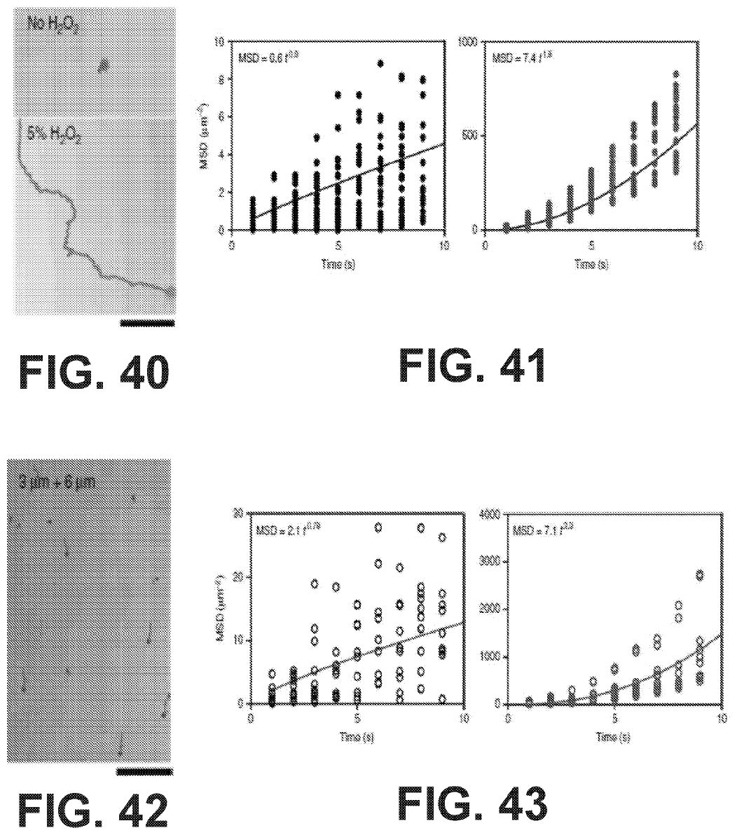

The velocity of Pt-motors prepared from 6-μm beads was tested in the presence and absence of H₂O₂. FIG. 37 shows the effect of the concentration of H₂O₂ on the motion of 6-μm Pt-motors. In the absence of H₂O₂, motors were just vibrating due to the Brownian motion, and in the presence of H₂O₂, the average velocity of the motors increased at a rate of ˜0.7 μm/s for 1% increase of H₂O₂ concentration. The sensing protocol relies on monitoring the change in motor motion due to the LAMP amplicon reaction on the surface of motors using DNA capture probes through thermal hybridization. Thus it was necessary to test the effect of temperature and incubation time in H₂O₂ on the velocity of the prepared motors. Aliquots of the prepared motors were incubated at 45, 80, and 100° C. for 10 min. The results indicated that the velocity of the prepared motors decreased with the increase in the temperature and there was a 10% loss of motion at 45° C. when compared to control (incubated at 25° C. as room temperature) (FIG. 38). On the other hand, the prepared motors were stable in their motion with time of incubation in H₂O₂. FIG. 39 presents the motion of motors for 120 s in 5% H₂O₂ solution. In the presence of H₂O₂, the motors autonomously move in a self-propelled fashion that is in principle due to the consumption of H₂O₂ and generation of gas bubbles. To investigate the motion of the prepared Pt-motors, their motion trajectories in the presence and absence of H₂O₂ were recorded under a bright-filed light microscope and then analyzed by plotting the mean squared displacement (MSD) against time (t). MSD is known to be proportional to tα for scaling exponent α. It was found that the MSD versus time had a near linear dependence (α=0.9) when no H₂O₂ was added, as is the case for random diffusion (α=1), while in the presence of 5% H₂O₂ solution, α=1.8 indicating that the motor motion is caused by the catalytic activity of the surface PtNPs and differs from random diffusion (FIGS. 40, 41). To further confirm the catalytic nature of the in motor motion due to the LAMP amplicon reaction on the surface of motors using DNA capture probes through thermal hybridization. Thus it was necessary to test the effect of temperature and incubation time in H₂O₂ on the velocity of the prepared motors. To further confirm the catalytic nature of the prepared motors (i.e., move due to the decomposition of H₂O₂ by PtNPs), samples with a mixture of 6-μm motor (coated with PtNPs) and non-modified 3-μm beads (no PtNPs) in 5% H₂O₂ solution were tested and their motion was analyzed using bright field light microscopy and Image J software. The slope of MSD plot of the 3-μm beads and 6-μm Pt-motors suggests a fundamentally different mode of motion (FIGS. 41, 42).

### Development of Pt-Motor Tracking Cellphone System

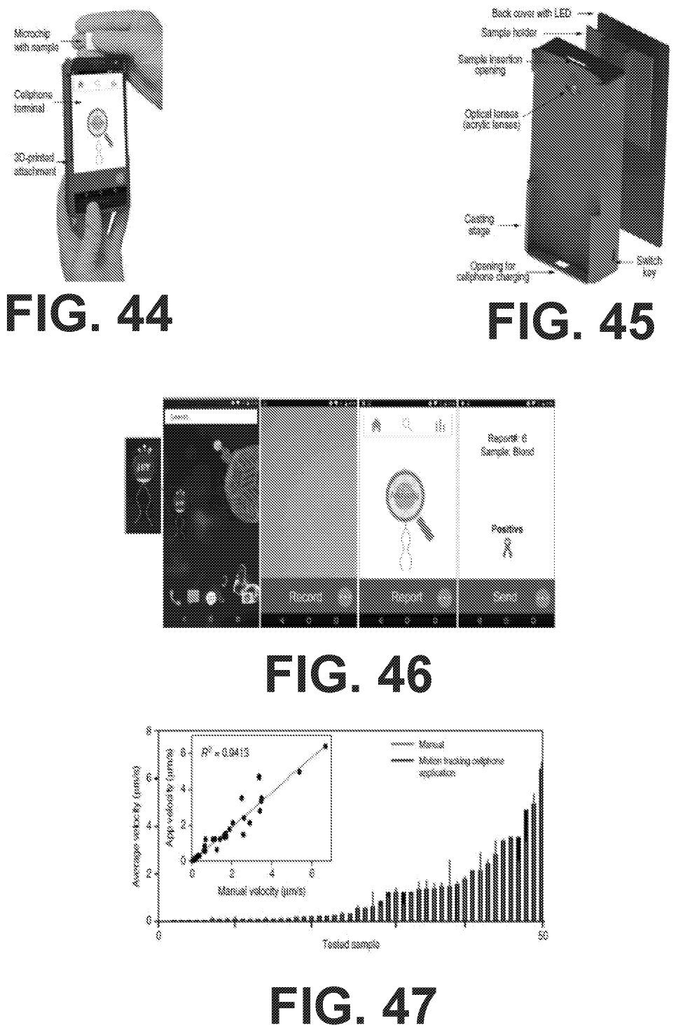

The cellphone system used in visualizing and tracking the motion of micromotors included an android terminal (XT1575, Motorola) modified with an optical attachment and a cellphone application on a single-channel microfluidic device (FIG. 44). The microchip used to test the motion of motors was prepared of poly(methylmethacrylate) (PMMA) substrate and a glass slide attached to each other using a 80-μm double-sided adhesive (DSA) machined with a laser cutter to fabricate a single microchannel with 2 mm width. The cellphone attachment was designed in Solidworks and three-dimensional (3D) printed with low-cost polylactic acid (PLA) material. The 3D printed enclosure housed a broadband white light-emitting diode (LED), a 3.3-V battery, a switch, and inexpensive optical lenses. The 3D construct also includes a sample holder to focus the sample between the two lenses and the cellphone camera (FIG. 45). The optical attachment and sample holder were custom-designed to facilitate chip insertion and positioning on the attachment through a simple slide-on mechanism and in a way that the chip remains in optimal focus without the need for manual focusing. The software on the cellphone was developed in Android Studio using OpenCV (ver. 3.1.0) libraries with a user-friendly interface to guide the user through the testing process (FIG. 46). The cellphone application records videos of samples, enumerates motors, automatically calculates the velocity of motors, and reports the results in <1 min. The developed application was able to record sample videos at a rate of 30 frames per second (fps) with a maximum effective field of view of 320×240 pixels for the cellphone used in this study (Motorola MotoX). The developed cellphone system was first calibrated using a micrometer scale. The performance of the developed system in visualizing, counting, and tracking the motion of Pt-motors was tested and correlated to the manual counts using bright-field microscopy. The results indicated a correlation coefficient of 0.9413 with a standard error of 0.3592 and a correlation coefficient of 0.9028 with a standard error of 0.3121 for motor velocity and enumeration, respectively. Furthermore, there was no statistical difference between the measurements (n=50) from the motion tracking application and the manual count performed by bright-field microscopy (P>0.05, paired t test) and with 95% confidence interval (CI) of −0.1564 to 0.04814 and −0.1491 to 0.02913 for motor velocity and enumeration, respectively (FIG. 47).

### LAMP-Reaction and Validation of the CALM System

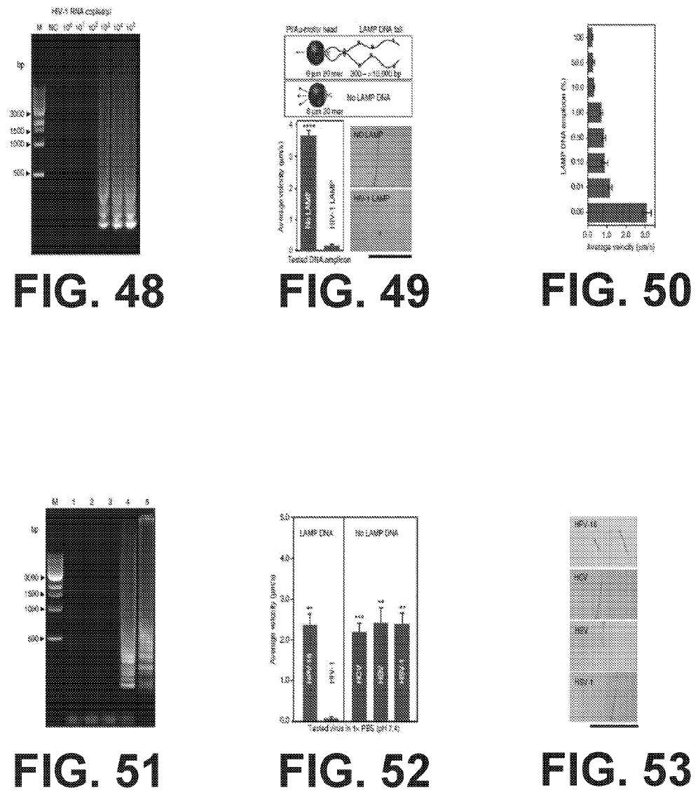

Reverse transcription-loop mediated isothermal amplification (RTLAMP) was performed using a set of four primers that target gag gene of HIV-1 (Table 2) following the standard protocol. Different concentrations of HIV-1 RNA template were prepared and used as a target in LAMP reaction. The amplification product was characterized using agarose gel electrophoresis (FIG. 48). The results indicated the formation of large-sized DNA amplicons appeared as ladder-like patterns with many bands (>320 bp in size) and the amount of these amplicons was proportional to the concentration of HIV-1 RNA.

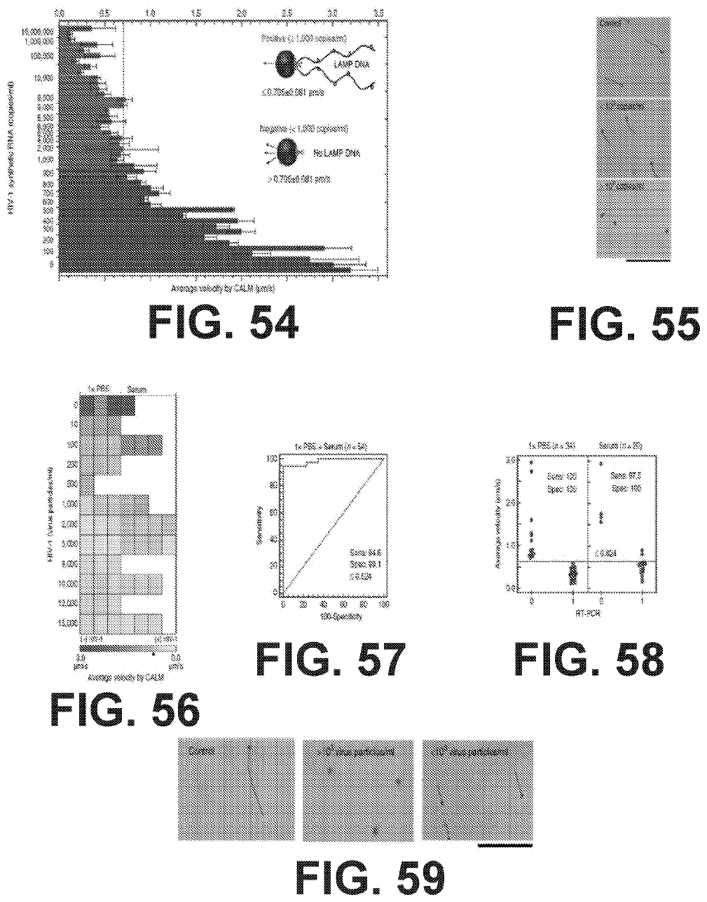

To detect the motion of motors in the presence and absence of HIV-1 LAMP amplicons, the motors were mixed with LAMP amplicons and allowed to hybridize at 80° C. The formed motor—LAMP DNA assemblies were tested in 5% H₂O₂ solution. In the presence of HIV-1 LAMP amplicons, the velocity of the motors (n=30) was significantly (P<0.0001, unpaired t test) decreased by 95.26% compared to control where no HIV-1 LAMP amplicons were added (only Pt motors) (FIG. 49). Subsequently, different dilutions of the target LAMP amplicons were allowed to hybridize with motors and the motion of the formed motor—DNA amplicon assemblies was tested in 5% H₂O₂ solution using the developed cellphone motion tracking system (FIG. 50). The results indicated that the presence of LAMP amplicons, even at a very low concentration of 3.394±0.245 ng/μl, reduced the velocity of motors compared to control (no LAMP amplicons), considering signal-to-noise ratio=3. The recovery of motor motion after releasing the target LAMP amplicons was tested by incubating the formed LAMP DNA-motor assemblies at 90° C. for 30 s. The released amplicons were separated from the motors by centrifugation at 6000×g for 5 min and tested in 5% H₂O₂ solution. There was a 75.52% recovery for the velocity of the tested motors. In addition, the response of the CALM system to non-target viruses was tested using different sexually transmitted RNA and DNA viruses commonly exist with HIV infection including hepatitis C virus (HCV), hepatitis B virus (HBV), herpes simplex virus type 1 (HSV-1), and human papillomavirus type 16 (HPV-16). The results of LAMP reaction confirmed that the DNA amplicons are only formed in the presence of the target HIV-1 and no visible amplification was observed with other tested viruses on agarose gel (FIG. 51). Furthermore, the average velocity of motors in the presence of the amplification products of non-target viruses (i.e., LAMP reaction products generated with HCV, HBV, and HSV-1) was not significantly (P>0.05, unpaired t test) different than control (no amplicons) samples and was at least three-folds higher than the average velocity of HIV-1 samples (FIGS. 52, 53). To further confirm the specificity of our system in the presence of the non-target amplicons or contamination, the motors were challenged with non-target LAMP amplicons generated from HPV-16 using specifically designed primers against envelop (E)-1 gene and at amplification temperature of 60° C. for 30 min. There was no significant change (P>0.05, unpaired t-test) in the velocity of motors (n=25) compared to control (no amplicons) in the presence of non-target amplicons confirming the high specificity of the developed CALM system for HIV-1 testing (FIGS. 52, 53).

### HIV-1 Detection Using the CALM System

The efficiency and reliability of the developed CALM system in HIV-1 detection was evaluated using PBS (1×PBS, pH 7.4) and serum samples spiked with HIV-1 and patient plasma samples. The developed system can qualitatively differentiate between samples with viral loads below (i.e., negative sample) and above (i.e., positive sample) a clinically relevant threshold value of 1000 copies/ml as recommended by the World Health Organization (WHO). To establish the motor velocity that corresponds to the threshold virus concentration of 1000 particles/ml, the system was first calibrated using 1×PBS samples (n=48) spiked with different concentrations of stabilized synthetic HIV-1 RNA (0-10⁷ copies/ml). The prepared samples were amplified using LAMP and the generated amplicons were allowed to interact with motors for target capture and detection using the CALM system. The results demonstrated an average velocity of 0.705±0.082 μm/s for samples with 1000 copies/ml and there was a significant difference (P<0.0001, unpaired t test) between the average velocity of samples spiked with target RNA concentrations below and above the threshold value of 1000 copies/ml (FIGS. 54, 55). Accordingly, the cellphone system was calibrated using this velocity value of 0.705±0.082 μm/s to allow qualitative testing of PBS and serum samples spiked with virus particles (n=54) (FIGS. 56-59). The qualitative results obtained by the CALM system compared to the standard quantitative real-time PCR (RT-PCR) technique are presented as heatmap in FIG. 56. In addition, the receiver operating characteristic analysis (n=54) showed that the CALM system has a sensitivity of 94.6% with a CI of 81.8-99.3% and a specificity of 99.1% with a CI of 80.5-100% at the threshold concentration of 1000 particles/ml. The area under the curve (AUC) was 0.984 with a binomial exact CI ranging from 0.905 to 1.00 and significance level P (area=0.5)<0.0001 (FIG. 57). The vertical scatter plot analysis showed that the accuracy of the CALM system in correctly classifying PBS (n=34) and serum (n=20) samples spiked with HIV as positive and negative were 100% and 90%, respectively (FIG. 58). The specificity and reliability of the developed CALM system was evaluated using serum samples spiked with HIV-1 and non-target viruses of HCV, HBV, and HSV-1. The results showed that the velocity of motors (n=30) significantly (P<0.01, unpaired t test) decreased in the presence of the HIV-1, while in the presence of non-target viruses the velocity of motors was not statistically different (P>0.05, unpaired t test) than HIV-free control samples. In addition, the performance of the CALM system in identifying HIV-infected patient plasma samples compared to the results obtained by iSCA assay, which is a quantitative real-time PCR assay with single-copy sensitivity targeting a highly conserved region of integrase in the HIV-1 pol gene widely used in clinical diagnosis of HIV infection and ART monitoring, were evaluated (Table 3). A 100% accordance was observed between the CALM system and iSCA assay in classifying patient plasma samples as positive (1000 virus particles/ml) and negative (<1000 virus particles/ml).

TABLE 3
CALM system evaluation using HIV-
infected patient serum samples

| Sample | iSCA assay | CALM system | |
|---|---|---|---|
| no.^{a} | (copies/ml)^{b} | (negative/positive) | |
| 1 | 0 | Negative | (2.675 ± 0.424 μm/s) |
| 2 | 0 | Negative | (2.079 ± 1.014 μm/s) |
| 3 | 375 | Negative | (1.335 ± 0.144 μm/s) |
| 4 | 540 | Negative | (0.913 ± 0.150 μm/s) |
| 5 | 19,958 | Positive | (0.141 ± 0.014 μm/s) |
| 6 | 136,366 | Positive | (0.219 ± 0.041 μm/s) |

^{a}Sample 5 was prepared by diluting sample 6 in 4;
^{b}iSCA assay is a quantitative real-time PCR assay with single-copy sensitivity targeting a highly conserved region of intergrase in the HIV-1 pol gene.

From the above description, those skilled in the art will perceive improvements, changes and modifications. Such improvements, changes and modifications are within the skill of one in the art and are intended to be covered by the appended claims.

What is claimed is:

1. A system comprising:
a sample testing unit, configured to house a sample, comprising:
a plurality of motor structures configured for self-propulsion based on a presence or an absence of a target analyte in the sample, each of the plurality of motor structures comprising:
a catalytic motor-like micro/nanoparticle; and
an attached functional material specific for the target analyte attached to the catalytic motor-like micro/nanoparticle;
a plurality of beads configured to experience a motion behavior based on the self-propulsion of the plurality of motor structures;
an optical recording unit comprising an optical arrangement configured to detect the motion behavior of the beads in the sample testing unit, wherein the motion behavior is indicative of the presence or the absence of the target analyte.

2. The system of claim 1, wherein the plurality of beads are modified with the plurality of motor structures to make a plurality of bead-motor structure complexes.

3. The system of claim 1, wherein the optical component further comprises a handheld device comprising a processor and configured for display of a visualization to determine the presence or the absence of the target analyte.

4. The system of claim 3, wherein the handheld device is a cellphone or a tablet computing device.

5. The system of claim 3, wherein the sample testing unit comprises an attachment for the handheld device and a device configured to house the sample and fit within the attachment,
wherein the handheld device is configured to utilize the optical component to detect the motion behavior of the beads within a portion of the device.

6. The system of claim 5, wherein the device comprises a microchip with at least one channel for loading the sample with the plurality of beads and the plurality of motor structures.

7. The system of claim 6, wherein the microchip facilitates the display of the visualization to determine the presence or the absence of the target analyte.

8. The system of claim 1, wherein the handheld device is configured to create the visualization as a video of the motion behavior.

9. The system of claim 1, wherein the beads are microbeads, each comprising a detectable color, a detectable size, and/or a detectable shape.

10. The system of claim 9, wherein each of the microbeads comprises a polymer material, a glass material, a metal material, and/or a metallic material.

11. The system of claim 1, wherein at least one of the catalytic motor-like micro/nanoparticles converts a chemical signal from the attached functional material into mechanical motion by at least one of self-electrophoresis, self-diffusiophoresis, or bubble-thrust.

12. The system of claim 1, wherein at least one of the catalytic motor-like micro/nanoparticles comprises Au, Cu, Fe, Pd, Zn, Cd, Ag, and/or Pt.

13. The system of claim 1, wherein at least one of the catalytic motor-like micro/nanoparticles comprises a spherical shape, a wire shape, a rod shape, a tube shape, and/or a helix shape.

14. The system of claim 1, wherein the functional material comprises an antibody, a nucleic acid amplicon, a DNA probe, an RNA probe, an aptamer, a protein, an intact virus, a vesicle, and/or a cell.

15. The system of claim 1, wherein the sample is a biological sample, a chemical sample, or an environmental sample.

16. A method comprising:
loading a sample into an optical attachment of a handheld device comprising a processor, wherein the sample comprises a plurality of motor structures configured for self-propulsion based on a presence or an absence of a target analyte in the sample and a plurality of beads;
determining, by the handheld device, an initial motion characteristic of the plurality of beads within the sample; and
tracking, by the handheld device, a change from the initial motion characteristic of the plurality of beads within the sample,
wherein the change from the initial motion characteristic is based on the presence or the absence of the target analyte in the sample.

17. The method of claim 16, wherein the change from the initial motion characteristic is a change in a velocity of the initial motion.

18. The method of claim 16, further comprising providing, by the handheld device, a diagnosis based on the presence of the absence of the analyte determined due to the change from the initial motion characteristic.

19. The method of claim 18, wherein the diagnosis is provided in a report related to the target analyte,
wherein the report comprises a concentration of the target analyte in the sample.

20. The method of claim 16, wherein the plurality of beads are modified with the plurality of motor structures to make a plurality of bead-motor structure complexes.
